(19) United States
(12) Patent Application Publication
(10) Pub. No.: US 2023/0407309 A1
(43) Pub. Date: Dec. 21, 2023

(54) ANTISENSE OLIGOMERS FOR TREATMENT OF DISEASE

(71) Applicants: MONASH UNIVERSITY, Clayton, Victoria (AU); MURDOCH UNIVERSITY, Murdoch, Western Australia (AU)

(72) Inventors: Stephen Wilton, Peppermint Grove (AU); May Aung-Htut, Willetton (AU); Merlin Christopher Thomas, Ashburton (AU); Raelene Jane Pickering, Cheltenham (AU)

(21) Appl. No.: 17/909,947

(22) PCT Filed: Mar. 22, 2021
(86) PCT No.: PCT/AU2021/050261

§ 371 Date: Sep. 7, 2022

(51) Int. Cl. C12N 15/113; A61P 31/14

(52) U.S. Cl. CPC C12N 15/1137; C12N 2310/11; A61P 31/14

(57) ABSTRACT

An isolated or purified antisense oligomer for modifying pre-mRNA splicing in the Angiotensin Converting Enzyme 2 (ACE2) to modulate splicing of the ACE2 gene transcript or part thereof which has a modified backbone structure and sequences with at least 75% sequence identity to such antisense oligomers and which have a modified backbone structure.

### FIELD OF THE INVENTION

The present invention relates to a method for the modulation of the splicing of pre-mRNA coding for Angiotensin Converting Enzyme 2 (ACE2) or part thereof, using splice-switching antisense oligonucleotides (AONs) to induce the generation of novel ACE2 splice variants encoding soluble ACE2 isoforms, and to methods of treatment of ACE2-related disorders using said modulators.

### BACKGROUND ART

Angiotensin Converting Enzyme 2 (ACE2; EC 3.4.17.23) is a 92-kDA type 1 integral membrane glycoprotein belonging to the exopeptidase superfamily. ACE2 is expressed and active in most tissues.

The human ACE2 gene is found in the Xp22.2 region of the human X chromosome. It comprises 18 exons and 17 introns, and a 5′ flanking region that regulates its transcription.

Human ACE2 protein is composed of a globular extracellular domain (residues 18-740), a single-pass transmembrane domain (residues 741-761) and a short cytoplasmic tail (residues 762-805). ACE2 is N-glycosylated at Asn53, Asn90, Asn103, Asn322, Asn432, Asn546, and Asn690.

The Severe Acute Respiratory Syndrome-related Coronaviruses (including SARS-CoV and SARS-CoV-2), HNL63-CoV (NL63-S) and the SARS-like WIV1-CoV, bind to the ectodomain of ACE2, at a site distinct and distant from its deeply-recessed catalytic domain. The affinity of the SARS-CoV-2 for ACE2 appears to be greater than that of other CoV including SARS-CoV, possibly contributing to its higher transmissibility in humans.

The ectodomain of ACE2 contains a single metallopeptidase domain that functions as a terminal carboxypeptidase, for example, cleaving the C-terminal phenylalanine from Ang II (1-8) to generate Ang (1-7). Genetic depletion of ACE2 results in increased levels of Ang II, and decreased levels of Ang-(1-7). As such, ACE2 is considered to be a key regulatory enzyme in the renin-angiotensin-aldosterone system (RAAS).

The RAAS is a homeostatic pathway that is implicated in the development and progression of many common diseases and disease processes. Inhibition of the RAAS with angiotensin-converting enzyme (ACE) inhibitors, or angiotensin II receptor type 1 (AT1R) blockers (inhibitors) is widely used for the management of many diseases and/or conditions including hypertension, cardiovascular disease (CVD), heart failure, chronic kidney disease (CKD), and diabetic complications. RAAS inhibition has also been shown to have benefits in preventing diabetes, in neuroprotection, modifying the growth of certain cancers and even in ageing, with genetic deletion of AT1R conferring longevity in mice. Activation of the RAAS is known to be an important mediator of atherosclerosis, chronic heart disease, chronic kidney disease, hypertension, lung disease and coronavirus infection.

The C-terminal of ACE2, incorporating residues 614-805, covering the non-catalytic extracellular, transmembrane, and intracellular domains of ACE2, shows 47.8% sequence identity with collectrin, a protein without enzymatic activity that is involved in the process of vesicle transport and membrane fusion. Consequently, ACE2 is considered to be a fusion protein, with 19-613 being ACE-like, and 614-805 being collectrin-like.

The cytoplasmic tail of ACE2 also contains integrin and calmodulin-binding sites. The cytosolic domain serves as trafficking adaptor for the large amino acid transporter B(O)AT1, transferring it to the apical membrane of epithelial cells in the intestine. The uptake of coronaviruses into cells is also dependent on internalisation mediated by the cytosolic tail of ACE2.

Most ACE2 is membrane-anchored. However, constitutive and inducible shedding of the ectodomain can occur under the influence of sheddases. Low levels of C-truncated ACE2 isoforms, generated by shedding, are naturally found in bloodstream, urine, bronchoalveolar fluids and saliva. This soluble isoform of ACE2 lacks the membrane anchor and cytoplasmic tail.

ACE2 is implicated in the development and progression of lung disease. Ace2 KO mice show enhanced lung injury in response to various models of lung disease and the injury phenotype can be rescued by reintroducing ACE2. In particular, the pathology of Acute Respiratory Distress Syndrome (ARDS) is amplified in the absence of ACE2 and a decline in lung ACE2 is prognostic of poor clinical outcomes. Recombinant soluble ACE2 has beneficial effects in experimental models of acute and chronic lung injury, and has been studies in clinical trials in patients with ARDS.

ACE2 plays an important role in atherosclerotic plaque development. We have previously shown that genetic deficiency of Ace2 is associated with increased plaque accumulation, comparable to that observed following angiotensin II infusion. ACE2 expression is reduced in established atherosclerotic plaques and in pro-atherosclerotic states, such as diabetes. Methods to increase circulating soluble ACE2 reduce atherosclerosis in this model.

Activation of the RAAS is known to be a key mediator of hypertension, and interventions to block RAAS activation are among the most widely used of all blood pressure lowering agents. The antihypertensive efficacy of these agents is partly mediated by their ability to reduce Ang II or its signalling. However, the antihypertensive effects of conventional RAS blockade are also partly determined by the ability of both ACE inhibitors and angiotensin receptor blockers (ARBs) to increase circulating levels of Ang(1-7). Given that the major source of Ang(1-7) in the vasculature is ACE2, this data suggests that ACE2 influences not only the development of hypertension, but also potentially the response to its treatment. ACE2 and the RAAS are also implicated in the pathogenesis of central hypertension. In the spontaneously hypertensive rats, ACE2 expression is reduced in the rostral ventrolateral medulla (RVLM), and persistent overexpression of ACE2 in the RVLM results in a significant attenuation of high blood pressure.

In the heart, ACE2 represents the primary pathway for the metabolism of Ang II. ACE2 deficiency in mice results in early cardiac hypertrophy and accelerates adverse post-myocardial infarction ventricular remodelling. In some models, ACE2 deficiency also results in progressive cardiac fibrosis with aging and/or cardiac pressure overload. Again, these changes are reversed following treatment with recombinant ACE2, ACE inhibitors or AT₁R blockers, suggesting that the balance of the RAAS in the heart is an important driving factor for progressive cardiac disease.

In the diabetic kidney and other states associated with renal damage and activation ACE2 plays an important role, as ACE2 deficient mice have accelerated renal damage. Renal damage in experimental diabetes is reduced by inventions increasing circulating soluble ACE2

The RAAS also has a number of metabolic functions. ACE2 deficiency is associated with insulin resistance and impaired glucose homeostasis, which is aggravated under high-fat diets. ACE2 deficiency is associated with increased lipid accumulation in skeletal muscle and liver.

It is against this background that the present method of using exon-skipping AONs for the modulation of ACE2 splicing, towards the preferred generation of soluble ACE2 over full length ACE2, is described.

The above discussion of the background art is intended to facilitate an understanding of the present invention only. The discussion is not an acknowledgement or admission that any of the material referred to is or was part of the common general knowledge as at the priority date of the application.

### SUMMARY OF INVENTION

Broadly, according to one form of the invention, there is provided an isolated or purified AON or combination of AONs that is used to modulate alternative splicing of pre-mRNA gene transcript coding for the Angiotensin Converting Enzyme 2 (ACE2) or part thereof. The purified AON preferably has a modified backbone structure. There is also provided sequences with at least 75%, 80%, 85%, 90% or at least 95% sequence identity to such antisense oligomers and which have a modified backbone structure.

In one aspect of the invention, there is provided an AON of 10 to 50 nucleotides comprising a targeting sequence complementary to a region near or within an intron of the ACE2 pre-mRNA.

In one aspect of the invention, there is provided an AON of 10 to 50 nucleotides comprising a targeting sequence complementary or adjacent to a splice site of the ACE2 pre-mRNA.

Because factors such as RNA secondary structure, competition between AONs and SR proteins, heterogeneous nuclear ribonucleoproteins (hnRNPs), and/or other elements that make up the spliceosome can affect the action of AONs, AONs directed at the crucial acceptor or donor splice sites will not always alter splicing. Consequently, in one aspect of the invention, there is provided an AON of 10 to 50 nucleotides comprising a targeting sequence complementary or adjacent to cis-acting RNA elements in the pre-mRNA of ACE2 that act as enhancers or silencers that, when bound by an element of the splicosome (e.g. protein-splicing factors, uRNA, IncRNA), modulates the splicing of a nearby exon.

In one aspect of the invention, there is provided an AON of 10 to 50 nucleotides comprising a targeting sequence complementary to ACE2 pre-mRNA which modulates secondary structure of said mRNA to influence splice site selection.

In one form of the invention, there is provided an isolated or purified AON for inducing exclusion (also known as skipping) of one or more exonic sequences in the ACE2 gene transcript or part thereof.

In one form of the invention, the AON is chemically-modified to prevent degradation of the pre-mRNA-AON complex, including but not limited to phosphorodiamidate morpholino oligomers (PMO), thio-morpholino oligonucleotides (TMO), 2′-O-Methyl (2′-O-Me) phosphorothioate (PTO) oligonucleotides and 2′-O-methoxyethyl (2′-MOE) PTO oligonucleotides, locked nucleic acid (LNA) modified AONs, thermostable twisted intercalating nucleic acid (TINA) and peptide nucleic acids (PNAs).

In one form of the invention, AONs are conjugated to moieties to increase their delivery. The moieties include, but not limited to, cell-penetrating peptides (CPPs), vivo-morpholinos (VMO) or peptide phosphorodiamidate morpholino oligomers (PPMO).

Preferably, the AON is selected from the group comprising the sequences set forth in Table 3, and combinations, derivatives or cocktails thereof. Preferably, the AON is selected from the list comprising: SEQ ID NO: 1-31, more preferably SEQ ID NO: 5, 6, 9 or 11. More preferably, the AON is SEQ ID NO: 6 and 9 or SEQ ID NO: 6 and 11.

The AON of the invention may be an AON capable of binding to a putative mRNA splicing site target site selected from a splice donor site, splice acceptor sites, splice enhancer sequences splice silencer sequences or sites that modulate the secondary structure of pre-mRNA. The target site may also include some flanking intronic sequences when the donor or acceptor splice sites are targeted.

More specifically, the AON may be selected from the group comprising of any one or more of SEQ ID NOs: 1-31, more preferably SEQ ID NO: 5, 6, 9 or 11, and/or the sequences set forth in any of Table 3and combinations, derivatives or cocktails thereof. More preferably, the AON is a combination of AONs; preferably a combination of SEQ ID NO: 6 and 9 or SEQ ID NO: 6 and 11. This includes sequences which can hybridise to such sequences under stringent hybridisation conditions, sequences complementary thereto, sequences containing modified bases, modified backbones, and functional truncations or extensions thereof which modulate pre-mRNA processing activity in a ACE2 gene transcript.

In certain embodiments, AONs may be 100% complementary to the target sequence, or may include mismatches, e.g., to accommodate variants, as long as a hetero-duplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

The invention extends also to a combination of two or more AONs capable of binding to a selected target to modulate alternative splicing of the ACE2 pre-mRNA, including a construct comprising two or more such AONs. The constructs may be used together for a combined AON-based therapy. The combination of AONs is preferably a combination of SEQ ID NO: 6 and 9 or SEQ ID NO: 6 and 11.

The invention extends, according to a still further aspect thereof, to cDNA or cloned copies of the AON sequences of the invention, as well as to vectors containing the AON sequences of the invention. The invention extends further also to cells containing such sequences and/or vectors.

There is also provided a method for manipulating splicing of a ACE2 gene transcript, the method including the step of:
a) providing one or more of the AONs as described herein and allowing the oligomer(s) to bind to a target nucleic acid site.

There is also provided a pharmaceutical, prophylactic, or therapeutic composition to treat, prevent or ameliorate the effects of a disease related to ACE2 expression in a subject, the composition comprising:
a) one or more AONs as described herein; and
b) one or more pharmaceutically acceptable carriers and/or diluents.

The composition may comprise about 1 nM to 1000 nM of each of the desired AON(s) of the invention. Preferably, the composition may comprise about 1 nM to 500 nM, most preferably between 1 nM and 10 nM of each of the AON(s) of the invention.

There is also provided a method to treat, prevent or ameliorate the effects of a disease associated with ACE2 expression, comprising the step of:
administering to the subject an effective amount of one or more AONs or pharmaceutical composition comprising one or more AONs as described herein.

In one form of the invention, AONs are co-administered with other agents that modulate the renin-angiotensin-aldosterone system (RAAS), including angiotensin receptor blockers and ACE inhibitors and recombinant ACE2.

In one form of the invention, AONs are co-administered with other agents that modulate coronavirus infectivity, including passive immunisation, active immunisation or antiviral therapy.

There is also provided the use of purified and isolated AONs as described herein, for the manufacture of a medicament to treat, prevent or ameliorate the effects of a disease associated with ACE2 expression and/or activity.

There is also provided a kit to treat, prevent or ameliorate the effects of a disease associated with ACE2 expression in a subject, which kit comprises at least an AON as described herein and combinations or cocktails thereof, packaged in a suitable container, together with instructions for its use.

Preferably the disease associated with ACE2 expression in a subject is coronavirus infection, lung diseases, atherosclerosis, chronic heart disease, chronic kidney disease or diabetes.

The subject with the disease associated with ACE2 expression may be a mammal, including a human.

Further aspects of the invention will now be described with reference to the accompanying non-limiting examples and drawings.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1a. Expression of soluble ACE2 protein in the cell culture media from CHO cells transfected with selected C-truncated ACE2 constructs on Western blotting, as detected by a polyclonal anti-ACE2 antibody (FIG. 1a-i) or a monoclonal anti-His antibody (FIG. 1a-ii).

FIG. 1b. Expression of soluble ACE2 protein in the cell culture media from CHO cells transfected with selected C-truncated mACE2 constructs, as detected by an anti-ACE2 ELISA. The bars show the mean ±SEM. n=6 per construct.

FIG. 1c. Catalytic activity of ACE2 in the culture media from CHO cells transfected with selected C-truncated mACE2 constructs, as detected by a quenched fluorescence ACE2 activity assay. The bars show the mean and the error bars show SEM. n=6 per construct.

FIG. 1d. Expression of soluble mACE2 protein in the culture media from CHO cells transfected with DNA mini-circles containing selected C-truncated mACE2 constructs, as detected by Western blotting using a polyclonal antiACE2 antibody.

FIG. 1e. Expression of soluble mACE2 protein in the culture media from CHO cells transfected with DNA minicircles encoding mACE2(19-615), as detected by anti-ACE2 ELISA. The bars show the mean and the error bars show SEM. n=6 per construct

FIG. 1f. The Ang II-induced expression of the inflammatory cytokine, MCP-1 in HAEC cells, is antagonised by pre-incubation with media from CHO cells overexpressing mACE2(19-615) and this protection was antagonised by the selective ACE2 inhibitor MLN4760. Data presented as mean ±SEM. Each column is a group of n=8.

FIG. 1g. The Ang II-induced expression of the adhesion molecule, ICAM-1 in HAEC cells, is antagonised by pre-incubation with media from CHO cells overexpressing mACE2(19-615) and this protection was antagonised by the selective ACE2 inhibitor, MLN4760.

FIG. 1h. Increased circulating ACE2 protein in the serum of mice four weeks after an intramuscular injection with a DNA minicircle encoding mACE2(19-615).

FIG. 1i. Increased circulating ACE2 activity in the serum of mice four weeks after an intramuscular injection with a DNA minicircle encoding mACE2(19-615).

FIG. 1j. Expression of human Y613L-hACE2(19-613) protein in 154 of cell media from transfected CHO cells, as detected by Western blotting, compared to vector transfected cells.

FIG. 1k. Catalytic activity of ACE2 in the culture media from CHO cells transfected with Y613L-hACE2(19-613), as detected by ACE2 activity assay. Recombinant Ace2 (1-740) is shown as a positive control and media from un-transfected CHO cells as the negative control.

FIG. 11. Plaque accumulation following infection of VeroE6 cells with SARS-CoV-2 is reduced following treatment with Y613L-ACE2 in a dose-dependent manner.

FIG. 2a. The constitutive splicing of ACE2 creates a fusion of exons 13-14-15. If this splicing pattern could be altered by using AONs targeting the donor and acceptor splice sites of exon 14, causing exon 14 skipping, it would generate a novel mRNA splice variant (Δ14 splice variant) encoding Y613L-ACE2 (19-613).

FIG. 2b. RT-PCR amplification plots showing the de novo expression of the Δ14 splice variant (arrow, green), induced 48 hours following treatment of human Caco-2 cells with (ii) H14A[−17+8] (100 nM) or (iii) a combination of H14A[−17+8] and H14D[+13−12] (50+50 nM). No expression of the Δ14 splice variant is observed in cells transfected with a non-target AON (100 nM, i). 18S is shown as an expression control (blue).

FIG. 2c. Expression of conventionally-spliced ACE2 mRNA containing exon 14 following treatment of human Caco-2 cells with combinations of AONs, specifically, H14D[+9−16] with H14A[−17+8] and H14D[+13−12] with H14A[−17+8], compared to a dose-control non-target AON. Data shows mean ±SEM. *p<0.01 vs dose control.

FIG. 2d. Expression of all ACE2 mRNA splice variants (i.e. ACE2 mRNA containing both exon 13 and exon 15) following transfection of human Caco-2 cells with ACE2-targeting AONs or non-target control AONs. Data shows mean ±SEM. *p<0.01 vs dose control. Data shows mean ±SEM.

FIG. 2e. Expression of conventionally-spliced ACE2 mRNA retaining exon 14 and all ACE2 mRNA splice variants (i.e. containing both exon 13 and exon 15) following transfection of human Caco-2 cells with AONs, specifically, H14D[+9−16] and H14A[−17+8], H14D[+13−12] and H14A[−17+8], or non-target control AONs. Data shows mean ±SEM. *p<0.01 vs dose control.

FIG. 2f. RT-PCR amplification plots showing the de novo expression of the Δ14 splice variant (green) 24 hours following treatment of human Caco-2 cells with a combination of AONs (i) H14D[+9−16] and H14A[−17+8]; 50+50 nM. No expression of the Δ14 splice variant is observed in cells transfected with a non-target AON (100 nM, ii). 18S is shown as an expression control (purple/blue).

FIG. 3a. Expression of conventionally-spliced ACE2 mRNA containing exon 14 and expression of all ACE2 mRNA splice variants containing both exon 13 and exon 15 following transfection of VeroE6 cells with ACE2-targeting AONs, specifically H14A[−17+8] and H14D[+13−12], or a non-target control AON. Data shows mean ±SEM. *p<0.01 vs dose control.

FIG. 3b. Expression of catalytically active soluble ACE2 in the cell media following transfection of VeroE6 cells with ACE2 targeting AONs, specifically H14A[−17+8] and H14D[+13−12] , H14A[−17+8] and H14D[+9−16], H14A[−6+19] and H14D[+13−12] or a non-target control AON, as measured by ACE2 activity in pooled media samples.

FIG. 3c. Expression of soluble ACE2 protein in the cell media following transfection of VeroE6 cells with ACE2 targeting AON, specifically H14A[−17+8] and H14D[+13−12] or a non-target control AON, as measured by ELISA. Data shows mean ±SEM. *p<0.01 vs dose control.

FIG. 3d. Adsorption of the SARS-CoV-2 spike glycoprotein (S1-subunit) onto the surface of VeroE6 cells following transfection with ACE2-targeting AONs, specifically H14A[−17+8] and H14D[+13−12] or a non-target control AON. The adsorption of GFP is shown as a negative control. Representative images are shown on the left. Data on the right shows mean ±SEM. *p<0.01 vs non-target control.

FIG. 4a. Expression of ACE2 mRNA splice variants in Caco-2 cells following transfection with 2′-O-Me PTO AONs (50 μM), as detected by one-step PCR using primers located in exon 13 (forward) and exon 15 (reverse).

FIG. 4b. Expression of ACE2 mRNA splice variants in Calu-3 cells following transfection with 2′-O-Me PTO AONs (50 nM) for 72 hours, as detected by one-step PCR using primers located in exon 13 (forward) and exon 15 (reverse).

FIG. 4c. The Median Tissue Culture Infectious Dose (TCID50) in the media of Calu-3 cells pre-treated with a vivo-morpholino formulation of H14A[−22+3] (SEQ ID NO: 6; 1 uM) for 72-hours, following by incubation with the SARS-CoV-2 (Vic01).

FIG. 5a. The reading frames of ACE2 transcript from exon 9 to 18, with and without exon 17 (the penultimate exon). Since exon 17 is an in-frame exon, removing exon 17 does not lead to a frameshift in the reading of exon 18.

FIG. 5b. Induction of different ACE2 mRNA splice variants, 24 hours following transfection of human fibroblasts with 50-200 μM 2′-O-Me PTO AONs targeting exon 17, as measured by one-step PCR, using primers located in exon 13 (forward) and exon 18 (reverse).

FIG. 5c. Induction of different ACE2 mRNA splice variants, 24 hours following transfection of a human keratinocyte cell line, HaCaT. with 50-200 μM 2′-O-Me PTO AONs targeting exon 17, as measured by one-step PCR, using primers located in exon 13 (forward) and exon 18 (reverse).

FIG. 5d. Time course analysis of exon 17 skipping after transfection of HaCaT cells with 50-200 μM of top three candidate AONs with 2′-O-Me PTO AONs targeting exon 17, as measured by one-step PCR, using primers located in exon 13 (forward) and exon 18 (reverse).

FIG. 5e. The expression of ACE2 mRNA splice variants in Caco-2 cells, 48 hours after treatment (1-3 μM) with a vivo morpholino formulation of M17A[+21+45] (SEQ ID NO: 9), using one-step PCR, using primers located in exon 13 (forward) and exon 18 (reverse).

FIG. 5f. Expression of ACE2 mRNA in the lungs of mice seven days after treatment with M17A[+21+45] (SEQ ID NO: 9) at a dose of 3 mg/kg delivered intratracheally in DI water, control AON or vehicle, as measured by real-time RT-PCR.

FIG. 5g. Expression of soluble ACE2 protein in the bronchoalveolar fluid of mice seven days after treatment with M17A[+21+45] (SEQ ID NO: 32) at a dose of 3 mg/kg delivered intratracheally in DI water, control AON or vehicle, as measured by ELISA.

FIG. 5h. The Median Tissue Culture Infectious Dose (TCID50) in the media of Calu-3 cells pre-treated with a vivo-morpholino formulation of H17A[+21+45] (SEQ ID NO: 9; 1 uM) for 72-hours, following by incubation with the SARS-CoV-2 (Vic01).

FIG. 6a. Expression of ACE2 mRNA splice variants in Calu-3 cells following treatment with vivo-morpholino AONs, H17A[+21+45] or H14A[−22+3] or both (1 μM each), as detected by one-step PCR using primers located in exon 13 (forward) and exon 15 (reverse).

FIG. 6b. Expression of ACE2 mRNA splice variants in Caco-2 cells following treatment with vivo-morpholino AONs, H17A[+21+45], H14A[−22+3], both H17A[+21+45] and H14A[−22+3], or a control oligonucleotide (1 μM each), as detected by one-step PCR using primers located in exon 13 (forward) and exon 15 (reverse).

FIG. 6c. Expression of ACE2 mRNA splice variants in Caco-2 cells following treatment with vivo-morpholino AONs, H17A[+21+45] or H14A[−22+3] or both (1 μM each), as detected by one-step PCR, using primers located in exon 13 (forward) and exon 18 (reverse).

FIG. 6d. The Median Tissue Culture Infectious Dose (TCID50) in the media of Calu-3 cells pre-treated with a combination of vivo-morpholino formulation of H17A[+21+45] and H14A[−22+3] (0.5 μM each) for 72-hours, following by incubation with the SARS-CoV-2 (Vic01).

FIG. 7. Sequences of the present application.

### DESCRIPTION OF INVENTION

### Detailed Description of the Invention

Antisense oligonucleotides (AON) are short, synthetic, antisense, modified strands of DNA or RNA that can selectively hybridise to pre-RNA/mRNA through Watson-Crick base pairing and selectively modulate the function of the target RNA.

When AONs are used to modulate alternative splicing of mRNA, they are often referred to as splice-switching oligonucleotides (SSO). In the present invention, the terms AON and SSO may be used interchangeably. AONs base-pair with a pre-mRNA and disrupt the normal splicing repertoire of the transcript by blocking the RNA-RNA base-pairing or protein-RNA binding interactions that occur between components of the splicing machinery and the pre-mRNA. AONs can induce “skipping” of selected exons and/or retention of intronic sequences to modulate the product of translation. This can be achieved by targeting splice sites directly or by targeting cis-acting sequences involved in enhancing or silencing splicing by modulating binding of specific proteins or altering secondary structure of the pre mRNA.

Therapeutic AONs may be used for the treatment of genetic disorders, to skip faulty or misaligned sections allowing for the generation of internally deleted, but now functional protein as a therapy.

Alternative splicing is not currently recognized as an important layer of post-transcriptional gene regulation for ACE2. Transcription sometimes does produce alternatively spliced variants naturally, the majority differing by the 5′ extension of exon 1. However, as these sequences are noncoding RNA the functional significance is unclear. Notably exon 14 is not naturally subject to alternative splicing.

The present invention uses AONs to manipulate the splicing pattern of ACE2 pre-mRNA, resulting in the generation of novel splice variants encoding soluble ACE2 isoforms that are enzymatically functional and/or that act as a decoy receptor to antagonise the binding of coronaviruses to endogenous ACE2.

Notably, to date no common ACE2 polymorphisms have been found at these splice sites. The ACE2 sequence is highly conserved, even between species. Therefore personalisation or individualised sequence modification is not required, unlike the management of genetic disorders with exon skipping technologies.

The present invention provides an alternative method for the treatment, prevention or amelioration of the effects of diseases in which ACE2 is implicated in the development or progression, including but not limited to coronavirus infections, lung disorders, hypertension, heart disorders, kidney disorders, and diabetes, by developing AONs that modulate the splicing of ACE2 pre-mRNA or part thereof.

Broadly, according to one aspect of the invention, there is provided an isolated or purified AON for modulating pre-mRNA splicing in ACE2 gene transcript or part thereof. Preferably, there is provided an isolated or purified AON for inducing exon exclusion and/or intron retention in the ACE2 pre mRNA or part thereof. The purified AON preferably has a modified backbone structure. There is also provided sequences with at least 95% sequence identity to such antisense oligomers and which have a modified backbone structure.

The invention provides an AON capable of binding to a selected target on a ACE2 gene transcript to modulate pre-mRNA splicing in the ACE2 gene transcript or part thereof. For example, in one aspect of the invention, there is provided an AON of 10 to 50 nucleotides comprising a targeting sequence complementary to a region of the ACE2 pre-mRNA or part thereof associated with the binding of a protein involved in the regulation of splicing of mRNA.

TABLE 1a
Map of the human ACE2 gene

| | | | region | phase at | |
|---|---|---|---|---|---|
| Region | start | end | length | end | Coding for amino acids: |
| Exon 1 | 15,618,849 | 15,619,034 | 235 | 0 | MSSSSWLLLSLVAVTAAQSTIEEQAKTFL |
| | | | | | DKFNHEAEDLFYQSSLASWNYNTNITEE |
| | | | | | NVQNM |
| Exon 2 | 15,612,968 | 15,613,126 | 159 | 0 | NNAGDKWSAFLKEQSTLAQMYPLQEIQ |
| | | | | | NLTVKLQLQALQQNGSSVLSEDKSKR |
| Exon 3 | 15,610,352 | 15,610,445 | 94 | 1 | LNTILNTMSTIYSTGKVCNPDNPQECLLLE |
| | | | | | P |
| Exon 4 | 15,609,836 | 15,609,979 | 144 | 1 | GLNEIMANSLDYNERLWAWESWRSEVG |
| | | | | | KQLRPLYEEYVVLKNEMARAN |
| Exon 5 | 15,607,467 | 15,607,579 | 113 | 0 | HYEDYGDYWRGDYEVNGVDGYDYSRG |
| | | | | | QLIEDVEHTFEE |
| Exon 6 | 15,605,876 | 15,605,981 | 106 | 1 | IKPLYEHLHAYVRAKLMNAYPSYISPIGCL |
| | | | | | PAHLL |
| Exon 7 | 15,603,598 | 15,603,695 | 98 | 0 | GDMWGRFWTNLYSLTVPFGQKPNIDVT |
| | | | | | DAMVDQ |
| Exon 8 | 15,599,344 | 15,599,513 | 170 | 2 | AWDAQRIFKEAEKFFVSVGLPNMTQGF |
| | | | | | WENSMLTDPGNVQKAVCHPTAWDLGK |
| | | | | | GDF |
| Exon 9 | 15,596,212 | 15,596,438 | 227 | 1 | RILMCTKVTMDDFLTAHHEMGHIQYDM |
| | | | | | AYAAQPFLLRNGANEGFHEAVGEIMSLS |
| | | | | | AATPKHLKSIGLLSPDFQEDN |
| Exon 10 | 15,593,789 | 15,593,933 | 145 | 2 | ETEINFLLKQALTIVGTLPFTYMLEKWRW |
| | | | | | MVFKGEIPKDQWMKKWWEM |
| Exon 11 | 15,591,490 | 15,591,588 | 99 | 2 | KREIVGVVEPVPHDETYCDPASLFHVSND |
| | | | | | YSFI |
| Exon 12 | 15,590,324 | 15,590,446 | 123 | 2 | RYYTRTLYQFQFQEALCQAAKHEGPLHK |
| | | | | | CDISNSTEAGQKL |
| Exon 13 | 15,589,747 | 15,589,919 | 173 | 1 | FNMLRLGKSEPWTLALENVVGAKNMNV |
| | | | | | RPLLNYFEPLFTWLKDQNKNSFVGWSTD |
| | | | | | WSP |
| Exon 14 | 15,588,418 | 15,588,476 | 59 | 0 | Y₆₁₃ADQSIKVRISLKSALGDKA₆₃₂ |
| Exon 15 | 15,585,849 | 15,585,949 | 101 | 2 | YEWNDNEMYLFRSSVAYAMRQYFLKVK |
| | | | | | NQMILF |
| Exon 16 | 15,584,376 | 15,584,492 | 117 | 2 | GEEDVRVANLKPRISFNFFVTAPKNVSDII |
| | | | | | PRTEVEKAI |
| Exon 17 | 15,582,147 | 15,582,341 | 195 | 2 | RMSRSRINDAFRLNDN |
| | | | | | SLEFLGIQPTLGPPNQPPVSIWLIVFGVV |
| | | | | | MGVIVVGIVILIFTGIRDRK |
| Exon 18 | 15,580,028 | 15,580,136 | 981 | 0 | KKNKARSGENP |
| | | | | | YASIDISKGENNPGFQNTDDVQTSF |

TABLE 1b
DNA sequence of the human ACE2 gene

5′ UPSTREAM SEQUENCE...caacccaagttcaaaggctgataagagagaaaatctcatgaggaggtttt
EXON-1 = ENSE00003897519
AGTCTAGGGAAAGTCATTCAGTGGATGTGATCTTGGCTCACAGGGGACGATGTCAAGCTCTTCCTGGCTCCTT
CTCAGCCTTGTTGCTGTAACTGCTGCTCAGTCCACCATTGAGGAACAGGCCAAGACATTTTTGGACAAGTTTAA
CCACGAAGCCGAAGACCTGTTCTATCAAAGTTCACTTGCTTCTTGGAATTATAACACCAATATTACTGAAGAGA
ATGTCCAAAACATG
INTRON-gtgagttctcatggctctattgggc...tcaattttcttttctgtcatttcag
EXON-2 = ENSE00000894057
AATAATGCTGGGGACAAATGGTCTGCCTTTTTAAAGGAACAGTCCACACTTGCCCAAATGTATCCACTACAAG
AAATTCAGAATCTCACAGTCAAGCTTCAGCTGCAGGCTCTTCAGCAAAATGGGTCTTCAGTGCTCTCAGAAGA
CAAGAGCAAACGG
INTRON gtacgtttgtgaacattttagcatt...attgtttattatctttaatttgcag
EXON-3 = ENSE00000894058
TTGAACACAATTCTAAATACAATGAGCACCATCTACAGTACTGGAAAAGTTTGTAACCCA
GATAATCCACAAGAATGCTTATTACTTGAACCAG
INTRON 3-4-gtaggctactaatttttagtagtga...gtctttgtgtgctttgggataacag
EXON 4 = ENSE00000894058
GTTTGAATGAAATAATGGCAAACAGTTTAGACTACAATGAGAGGCTCTGGGCTTGGGAAAGCTGGAGATCTG
AGGTCGGCAAGCAGCTGAGGCCATTATATGAAGAGTATGTGGTCTTGAAAAATGAGATGGCAAGAGCAAATC
INTRON-gtaagtttgctgatctgtagaggtc...tctctttaaaaaaaaaaacaaacag
EXON 5 = ENSE00000894060
ATTATGAGGACTATGGGGATTATTGGAGAGGAGACTATGAAGTAAATGGGGTAGATGGCTATGACTACAGCC
GCGGCCAGTTGATTGAAGATGTGGAACATACCTTTGAAGAG
INTRON-gtaagcaaggaactgtacacacaaa...taaactttcttcctgggcttttcag
EXON 6 = ENSE00000894061
ATTAAACCATTATATGAACATCTTCATGCCTATGTGAGGGCAAAGTTGATGAATGCCTATCCTTCCTATATCAG
TCCAATTGGATGCCTCCCTGCTCATTTGCTTG
Intron-gtaagaagccccatgaattcttcgt...ctaacagattcttttttaaatatag
EXON 7 = ENSE00000894062
GTGATATGTGGGGTAGATTTTGGACAAATCTGTACTCTTTGACAGTTCCCTTTGGACAGAAACCAAACATAGA
TGTTACTGATGCAATGGTGGACCAG
Intron gtaggaaaaagagcccttaaaaact...aattaatactgttctcttttcccag
Exon 8 = ENSE00000894063
GCCTGGGATGCACAGAGAATATTCAAGGAGGCCGAGAAGTTCTTTGTATCTGTTGGTCTTCCTAATATGACTC
AAGGATTCTGGGAAAATTCCATGCTAACGGACCCAGGAAATGTTCAGAAAGCAGTCTGCCATCCCACAGCTTG
GGACCTGGGGAAGGGCGACTTCAG
Intron gtagtggggctgatacttacacaac...tttcatcttgtccattttcatgcag
Exon 9 = ENSE00000894064
GATCCTTATGTGCACAAAGGTGACAATGGACGACTTCCTGACAGCTCATCATGAGATGGGGCATATCCAGTAT
GATATGGCATATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGATTTTCAAGAAG
ACAATG
Intron gtatggacattttctcatggcttgt...ttatccttttctattttactttcag
EXON 10 = ENSE00000894065
AAACAGAAATAAACTTCCTGCTCAAACAAGCACTCACGATTGTTGGGACTCTGCCATTTACTTACATGTTAGAG
AAGTGGAGGTGGATGGTCTTTAAAGGGGAAATTCCCAAAGACCAGTGGATGAAAAAGTGGTGGGAGATGAA
Intron gtaagtcaatgaatatgcaatcagt...gtattgttttcttttctccccaaag
Exon 11 = ENSE00000894066
GCGAGAGATAGTTGGGGTGGTGGAACCTGTGCCCCATGATGAAACATACTGTGACCCCGCATCTCTGTTCCAT
GTTTCTAATGATTACTCATTCATTCG
Intron gtaaattacagttttcttgtttctg...ttttgttgctttgtctcctgtgcag
Exon 12 = ENSE00000894067
ATATTACACAAGGACCCTTTACCAATTCCAGTTTCAAGAAGCACTTTGTCAAGCAGCTAAACATGAAGGCCCTC
TGCACAAATGTGACATCTCAAACTCTACAGAAGCTGGACAGAAACTGTT
Intron gtaagaaatacctcaaaatgttgaa...ccctgaaccccctttttttgtgtag
Exon 13 = ENSE00000894068
CAATATGCTGAGGCTTGGAAAATCAGAACCCTGGACCCTAGCATTGGAAAATGTTGTAGGAGCAAAGAACAT
GAATGTAAGGCCACTGCTCAACTACTTTGAGCCCTTATTTACCTGGCTGAAAGACCAGAACAAGAATTCTTTTG
TGGGATGGAGTACCGACTGGAGTCCAT
Intron gtgagtacacccagttgacaagttt...cagtctgctatttctctttaatcag
ATGCAGACCAAAGCATCAAAGTGAGGATAAGCCTAAAATCAGCTCTTGGAGATAAAGCA
Intron gtgagtattctggacagtgaattga...cagtgtctttcttctgaatttgcag
EXON 15 = ENSE00000894070
TATGAATGGAACGACAATGAAATGTACCTGTTCCGATCATCTGTTGCATATGCTATGAGGCAGTACTTTTTAAA
AGTAAAAAATCAGATGATTCTTTTTGG
Intron gtgagttgatttgctgggttctcaa...agatgtttgttttgtttctctacag
EXON 16 = ENSE00000894071
GGAGGAGGATGTGCGAGTGGCTAATTTGAAACCAAGAATCTCCTTTAATTTCTTTGTCAC
TGCACCTAAAAATGTGTCTGATATCATTCCTAGAACTGAAGTTGAAAAGGCCATCAG
Intron gtgacattttactttcatctaaggg...tgctgttttttgcttttgcaaatag
EXON 17 = ENSE00003507229
GATGTCCCGGAGCCGTATCAATGATGCTTTCCGTCTGAATGACAACAGCCTAGAGTTTCTGGGGATACAGCCA
ACACTTGGACCTCCTAACCAGCCCCCTGTTTCCATATGGCTGATTGTTTTTGGAGTTGTGATGGGAGTGATAGT
GGTTGGCATTGTCATCCTGATCTTCACTGGGATCAGAGATCGGAAGAA
Intron-gtaagtggcctttcctagacttaac...aaggcaaccttgctctatttaacag
Exon 18 = ENSE00001334055
GAAAAATAAAGCAAGAAGTGGAGAAAATCCTTATGCCTCCATCGATATTAGCAAAGGAGAAAATAATCCAGG
ATTCCAAAACACTGATGATGTTCAGACCTCCTTTTAGAAAAATCTATGTTTTTCCTCTTGAGGTGATTTTGTTGT
ATGTAAATGTTAATTTCATGGTATAGAAAATATAAGATGATAAAGATATCATTAAATGTCAAAACTATGACTCT
GTTCAGAAAAAAAATTGTCCAAAGACAACATGGCCAAGGAGAGAGCATCTTCATTGACATTGCTTTCAGTATT
TATTTCTGTCTCTGGATTTGACTTCTGTTCTGTTTCTTAATAAGGATTTTGTATTAGAGTATATTAGGGAAAGTG
TGTATTTGGTCTCACAGGCTGTTCAGGGATAATCTAAATGTAAATGTCTGTTGAATTTCTGAAGTTGAAAACAA
GGATATATCATTGGAGCAAGTGTTGGATCTTGTATGGAATATGGATGGATCACTTGTAAGGACAGTGCCTGG
GAACTGGTGTAGCTGCAAGGATTGAGAATGGCATGCATTAGCTCACTTTCATTTAATCCATTGTCAAGGATGA
CATGCTTTCTTCACAGTAACTCAGTTCAAGTACTATGGTGATTTGCCTACAGTGATGTTTGGAATCGATCATGC
TTTCTTCAAGGTGACAGGTCTAAAGAGAGAAGAATCCAGGGAACAGGTAGAGGACATTGCTTTTTCACTTCCA
AGGTGCTTGATCAACATCTCCCTGACAACACAAAACTAGAGCCAGGGGCCTCCGTGAACTCCCAGAGCATGCC
TGATAGAAACTCATTTCTACTGTTCTCTAACTGTGGAGTGAATGGAAATTCCAACTGTATGTTCACCCTCTGAA
GTGGGTACCCAGTCTCTTAAATCTTTTGTATTTGCTCACAGTGTTTGAGCAGTGCTGAGCACAAAGCAGACACT
CAATAAATGCTAGATTTACACACTC
3′DOWNSTREAM-cttgtgcttacttatgtgctggggcttctttacgttttgtctgcttttca...

TABLE 2
Protein domains of the human ACE2 protein

| Residues | Domain |
|---|---|
| 1 . . . 17 | Signal peptide |
| 18 . . . 740 | Extracellular domain |
| 30 . . . 41 | Interaction with SARS-CoV spike glycoprotein |
| 82 . . . 84 | Interaction with SARS-CoV spike glycoprotein |
| 353 . . . 357 | Interaction with SARS-CoV spike glycoprotein |
| 614-805 | Collectrin-homology domain |
| 652 . . . 659 | Essential for cleavage by ADAM17 |
| 697 . . . 716 | Essential for cleavage by TMPRSS11D and TMPRSS2 |
| 741 . . . 761 | Trans-membrane region |
| 762 . . . 805 | Cytosolic tail |

In contrast to other AON based therapies, the present invention does not induce increased degradation of RNA via recruitment of RNase H, wherein the RNase H preferentially binds and degraded RNA bound in duplex to DNA of the ACE2 gene. Nor does it rely on hybridization of the AON to the ACE2 genomic DNA or the binding of AONs to mRNA to modulate the amount of ACE2 protein produced by interfering with normal functions such as replication, transcription, translocation and translation. Rather, the AONs are used to selectively modulate pre-mRNA splicing of an ACE2 gene transcript or part thereof and induce exon “skipping”. The strategy preferably reduces the expression of full length membrane-associated ACE2 and/or increases the generation of truncated soluble ACE2 isoforms that lack transmembrane or cytoplasmic domains.

According to a first aspect of the invention, there is provided AONs capable of binding to a selected target on a ACE2 gene transcript to modulate pre-mRNA splicing in a ACE2 gene transcript or part thereof. Broadly, there is provided an isolated or purified AON for inducing targeted exon exclusion/intron retention in a ACE2 gene transcript or part thereof.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells, refers to the purification of cells (e.g., fibroblasts, lymphoblasts) from a source subject (e.g., a subject with a polynucleotide repeat disease). In the context of DNA, mRNA or protein, “isolating” refers to the recovery of the DNA, mRNA or protein from a source, e.g., cells.

An AON can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing, including splice enhancers and splice silencers and sites determining the secondary structure of RNA that influence splicing. The target sequence may be within an exon or within an intron or spanning an intron/exon junction.

In certain embodiments, the AON has sufficient sequence complementarity to a target RNA (i.e., the RNA for which splice site selection is modulated) to block a region of a target RNA (e.g., pre-mRNA) in an effective manner. In exemplary embodiments, such blocking of ACE2 pre-mRNA serves to modulate splicing, either by masking a binding site for a splicosomal protein that would otherwise modulate splicing and/or by altering the structure of the targeted RNA. In some embodiments, the target RNA is target pre-mRNA (e.g., ACE2 gene pre-mRNA).

An AON having a sufficient sequence complementarity to a target RNA sequence to modulate splicing of the target RNA means that the AON has a sequence sufficient to trigger the masking of a binding site for a native protein that would otherwise modulate splicing and/or alters the three-dimensional structure of the targeted RNA.

Selected AONs can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the AON is selected from the group comprising SEQ ID NOS: 1-31, more preferably SEQ ID NO: 5, 6, 9 or 11, and/or the sequences set forth in Table 3. More preferably, the AON is SEQ ID NO: 6 and 9 or SEQ ID NO: 6 and 11.

In certain embodiments, the degree of complementarity between the target sequence and AON is sufficient to form a stable duplex. The region of complementarity of the AONs with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An AON of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphoro-diamidate morpholino oligomer (PMO) AONs described further herein, an optimum balance of binding stability and uptake generally occurs at lengths of 18-25 bases. Included are AONs (e.g., PMOs, PMO-X, PNAs, LNAs, TINA, 2′-O-Me) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49 or 50 bases.

In certain embodiments, AONs may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that splicing of the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-31, more preferably SEQ ID NO: 5, 6, 9 or 11 and/or the sequences set forth in Table 3. More preferably, the AON is SEQ ID NO: 6 and 9 or SEQ ID NO: 6 and 11.

More specifically, there is provided an AON capable of binding to a selected target site to modify pre-mRNA splicing in a ACE2 gene transcript or part thereof. The AON is preferably selected from SEQ ID NOS: 1-31, more preferably SEQ ID NO: 5, 6, 9 or 11, and/or the sequences set forth in any of Table 3. More preferably, the AON is SEQ ID NO: 6 and 9 or SEQ ID NO: 6 and 11.

The modification of pre-mRNA splicing preferably induces “skipping”, or the removal of one or more exons or retention of introns of the mRNA. The resultant protein is preferably of a shorter length when compared to the parent full-length ACE2 protein due to either internal truncation or premature termination. These C-truncated ACE2 proteins may be termed isoforms of the full length ACE2 protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (e.g. a nonsense sequence) due to a frame-shift. Alternatively, the exon skipping may induce the production of a prematurely terminated protein due to a disruption of the reading frame and presence of a premature termination of translation. The prematurely terminated protein may be the result of mRNA that is prematurely terminated (e.g. skipping of exons 14) or missense skip which provides an mRNA that contains the exon 14 mRNA, but which does not provide expression of the protein encoded by these exons.

Skipping individual exons may preferably disrupt the reading frame of the ACE2 transcript. This may introduce a missense coding sequence, that leads to protein dysfunction or degradation, or non-sense coding sequence leading to a premature termination codon resulting in the translation of a C-truncated protein or increased degradation of mRNA through nonsense mediated decay.

Skipping individual exons may preferably keep the reading frame intact. This will preferably lead to translation of an internally-truncated protein. The truncated protein or ACE2 isoform may have a completely ablated function, may have a reduced function or act as a decoy receptor.

Preferably, these truncated, or prematurely terminated proteins are lacking one or more functional domains involved in membrane retention and receptor internalisation.

For example, Exon 14 encodes the start of the non-catalytic collectrin-like domain of ACE2 and removing this exon may generate a soluble ACE2 protein, which could potentially act as a soluble decoy or competitive antagonist of coronavirus internalisation mediated via ACE2. Truncated, nonsense or prematurely terminated proteins may further lack an attachment or binding site for other factors, removal of which may lead to a reduction in interaction of the ACE2 protein with relevant signalling pathways.

The presence of internally truncated proteins (i.e. proteins lacking the amino acids encoded by one or more exons) is preferable. If the ACE2 catalytic activity is inhibited, there may be problems with elevation of ACE2 transcription as the body tries to compensate for the reduction in the total amount of ACE2 protein. In contrast, the presence of an internally truncated protein (preferably lacking one or more of the features of the complete ACE2 protein), should be sufficient to prevent elevated transcription, but still provide a therapeutic advantage due to an increase in soluble ACE2 and a reduction in the total amount of membrane-bound ACE2 protein.

The skipping process of the present invention, using AONs, may exclude (skip) an individual exon, or may result in skipping two or more exons at once.

The skipping process of the present invention, using AONs, may include retention of intronic sequences with or without directly skipping one or more exons.

The AONs of the invention may be a combination of two or more AONs capable of binding to a selected target to induce exon exclusion in a ACE2 gene transcript. The combination may be a cocktail of two or more AONs and/or a construct comprising two or more or two or more AONs joined together.

TABLE 3
Sequence of AONs used for modulation of the exon
splicing of human ACE2

| SEQ | | |
|---|---|---|
| ID NO | Name | Sequence (5′ to 3′) |
| 1 | H14D[+09 −16] | GUCGAGAACCUCUAUUUCGUCACUC |
| 2 | H14A[−06 +19] | UUAGUCUACGUCUGGUUUCGUAGUU |
| 3 | H14D[+13 −12] | UUAGUCGAGAACCUCUAUUUCGUCA |
| 4 | H14A[−17 +8] | AUAAAGAGAAAUUAGUCUACGUCUG |
| 5 | H14A[−25 −1] | CUGAUUAAAGAGAAAUAGCAGACUG |
| 6* | H14A[−22 +3] | CAUCUGAUUAAAGAGAAAUAGCAGA |
| 7 | H14A[−20 +5] | UGCAUCUGAUUAAAGAGAAAUAGCA |
| 8 | H14A[+22 +45] | GAGCUGAUUUUAGGCUUAUCCUCAC |
| 9* | H17A[+21 +45] | UUGUCAUUCAGACGGAAAGCAUCAU |
| 10 | H17A[−15 +10] | CCGGGACAUCCUAUUUGCAAAAGCA |
| 11* | H17A[−05 +20] | UGAUACGGCUCCGGGACAUCCUAUU |
| 12 | H17A[−10 +15] | CGGCUCCGGGACAUCCUAUUUGCAA |
| 13 | H17A[+56 +80] | CAAGUGUUGGCUGUAUCCCCAGAAA |
| 14 | H17A[+88 +112] | CCAUAUGGAAACAGGGGGCUGGUUA |
| 15 | H17A[+160 +184] | UCUGAUCCCAGUGAAGAUCAGGAUG |
| 16 | H17D[+10 −15] | GGAAAGGCCACUUACUUCUUCCGAU |
| 17 | H17A[+46 +70] | CUGUAUCCCCAGAAACUCUAGGCUG |
| 18 | H17A[+155 +179] | UCCCAGUGAAGAUCAGGAUGACAAU |
| 19 | H17A[+165 +189] | CGAUCUCUGAUCCCAGUGAAGAUCA |
| 20 | H17A[+113 +137] | CCAUCACAACUCCAAAAACAAUCAG |
| 21 | H17A[+138 +162] | AUGACAAUGCCAACCACUAUCACUC |
| 22 | H17A[−02 +18] | AUACGGCUCCGGGACAUCCU |
| 23 | H17A[+02 +21] | UUGAUACGGCUCCGGGACAU |
| 24 | H17A[+05 +24] | UCAUUGAUACGGCUCCGGGA |
| 25 | H17A[+08 +27] | GCAUCAUUGAUACGGCUCCG |
| 26 | H17A[+11 +30] | AAAGCAUCAUUGAUACGGCU |
| 27 | H17A[+14 +33] | CGGAAAGCAUCAUUGAUACG |
| 28 | H17A[+17 +36] | AGACGGAAAGCAUCAUUGAU |
| 29 | H17A[+20 +39] | UUCAGACGGAAAGCAUCAUU |
| 30 | H17A[+23 +42] | UCAUUCAGACGGAAAGCAUC |
| 31 | H17A[+26 +45] | UUGUCAUUCAGACGGAAAGC |

The invention further provides a method for modulating alterative splicing in a ACE2 gene transcript, the method including the step of:
providing one or more of the AONs as described herein and allowing the oligomer(s) to bind to a target nucleic acid site.

According to yet another aspect of the invention, there is provided a nucleic acid sequence target for modulating alterative splicing of ACE2 pre mRNA comprising the DNA equivalents of the nucleic acid sequences selected from the group consisting of SEQ ID NOs: 1-31 more preferably SEQ ID NO: 5, 6, 9 or 11, and/or the sequences set forth in any of Table 3 and sequences complementary thereto. More preferably, the combination of AONs is preferably a combination of SEQ ID NO: 6 and 9, or SEQ ID NO: 6 and 11.

Designing AONs to completely mask consensus splice sites may not necessarily generate a change in splicing of the targeted exon. Furthermore, the inventors have discovered that size or length of the AON itself is not always a primary factor when designing AONs. With some targets, AONs as short as 20 bases were able to induce some exon inclusion, in certain cases more efficiently than other longer (e.g. 25 bases) oligomers directed to the same exon.

The inventors have also discovered that there does not appear to be any standard motif that can be blocked or masked by AONs to redirect splicing. It has been found that AONs must be designed and their individual efficacy evaluated empirically for each gene target.

More specifically, the AON may be selected from those set forth in Tables 3.The sequences are preferably selected from the group consisting of any one or more of any one or more of SEQ ID NOs: 1-31, more preferably SEQ ID NO: 5, 6, 9 or 11, and combinations or cocktails thereof. The combination of AONs is preferably a combination of SEQ ID NO: 6 and 9, or SEQ ID NO: 6 and 11. This includes sequences which can hybridise to such sequences under stringent hybridisation conditions, sequences complementary thereto, sequences containing modified bases, modified backbones, and functional truncations or extensions thereof which possess or modulate pre-mRNA processing in an ACE2 gene transcript.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0).Thus, the AONs of the present invention may include oligomers that selectively hybridise to the sequences provided in Table 3, or SEQ ID NOs: 1-31, more preferably SEQ ID NO: 5, 6, 9 or 11. More preferably, the combination of AONs is preferably a combination of SEQ ID NO: 6 and 9, or SEQ ID NO: 6 and 11.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of AONs may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the AON. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the AON sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the AON, the greater the homology required to obtain selective hybridisation. Consequently, where an AON of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AON of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e., downstream effects) in a cell or a subject relative to the response caused by either no AON or a control compound. The terms “decreasing” or “decrease” refer generally to the ability of one or AONs or compositions to produce or cause a reduced physiological response (i.e., downstream effects) in a cell or a subject relative to the response caused by either no AON or a control compound.

Relevant physiological or cellular responses (in vivo or in vitro) will be apparent to persons skilled in the art, and may include increases in the exclusion of specific exons in ACE2-coding pre-mRNA, increases in soluble ACE2 or decreases in the expression of full length ACE2 protein in a cell, tissue, or subject in need thereof. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7, 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease such as coronavirus infection, lung disorders, chronic kidney disease, chronic heart disease hypertension and diabetes. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an AON may vary, as long as it is capable of binding selectively to the intended location within the pre-mRNA molecule. The length of such sequences can be determined in accordance with selection procedures described herein. Generally, the AON will be from about 10 nucleotides in length, up to about 50 nucleotides in length. It will be appreciated, however, that any length of nucleotides within this range may be used in the method. Preferably, the length of the AON is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “AON” refers to a linear sequence of nucleotides, or nucleotide analogs, that allows the nucleobase to hybridize to a target sequence in an RNA by Watson-Crick base pairing, to form an oligonucleotide:RNA heteroduplex within the target sequence. The terms “AON”, “AON”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Me PTO oligonucleotides, among other antisense agents known in the art.

Included are non-naturally-occurring AONs, or “oligonucleotide analogs”, including AONs or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-mRNA during duplex formation with the AONs, the AONs used in the method may be adapted to minimise or prevent cleavage by endogenous RNase H. This property is highly preferred, as the treatment of the RNA with the unmethylated oligomers, either intracellular or in crude extracts that contain RNase H, leads to degradation of the pre-mRNA:AON duplexes. Any form of modified AONs that is capable of by-passing or not inducing such degradation may be used in the present method. The nuclease resistance may be achieved by modifying the AONs of the invention so that it comprises partially unsaturated aliphatic hydrocarbon chain and one or more polar or charged groups including carboxylic acid groups, ester groups, and alcohol groups.

An example of AONs which when duplexed with RNA are not cleaved by cellular RNase H is 2′-O-Me derivatives. Such 2′-O-Me oligoribonucleotides are stable in a cellular environment and in animal tissues, and their duplexes with RNA have higher Tm values than their ribo- or deoxyribo- counterparts. Alternatively, the nuclease resistant AONs of the invention may have at least one of the last 3′-terminus nucleotides fluoridated. Still alternatively, the nuclease resistant AONs of the invention have phosphorothioate bonds linking between at least two of the last 3-terminus nucleotide bases, preferably having phosphorothioate bonds linking between the last four 3′-terminal nucleotide bases.

Increased splice-switching may also be achieved with alternative oligonucleotide chemistry. For example, the AON may be chosen from the list comprising: phosphoramidate or phosphorodiamidate morpholino oligomer (PMO); PMO-X; PPMO; peptide nucleic acid (PNA); a locked nucleic acid (LNA) a thiomorpholino (IMO); and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′-O-Me modified oligomer (2′-O-Me); 2′-O-methoxy ethyl (2′-MOE); 2′-fluoro, 2′-fluroarabino (FANA); unlocked nucleic acid (UNA); thermostable twisted intercalating nucleic acid (TINA), hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping AONs. Antisense oligonucleotide-induced splice modification of the human ACE2 gene transcripts have generally used either oligoribonucleotides, PNAs, 2′-O-Me or 2′-MOE modified bases on a phosphorothioate backbone. 2′-O-Me PTO AONs are generally used for oligo design, due to their efficient uptake in vitro when delivered as cationic lipoplexes. When alternative chemistries are used to generate the AONs of the present invention, the uracil (U) of the sequences provided herein may be replaced by a thymine (T) or ribonucleotides replaced with deoxyribonucleotides.

Included within the AONs of the present invention are non-naturally-occurring oligomers, or “oligonucleotide analogues,” including oligomers having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligomer analogues support bases capable of hydrogen bonding by Watson-Crick base pairing to standard polynucleotide bases, where the analogue backbone presents the bases in a manner to permit such hydrogen bonding in a sequence-specific fashion between the oligomer analogue molecule and bases in a standard polynucleotide (e.g., single-stranded RNA or single-stranded DNA). Preferred analogues are those having a substantially uncharged, phosphorus containing backbone.

Antisense oligonucleotides that do not activate RNase H can be made in accordance with known techniques (see, e.g., U.S. Pat. 5,149,797). Such AONs, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous AONs that do not activate RNase H are available. For example, such AONs may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl).For example, every other one of the nucleotides may be modified as described.

While the AONs described above are a preferred form of the AONs of the present invention, the present invention includes other oligomeric antisense molecules, including but not limited to oligomer mimetics such as are described below.

Specific examples of preferred AONs useful in this invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be AONs.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5- propynyluracil and 5-propynylcytosine.5-methylcytosine substitutions have been shown to increase nucleic acid duplex stability by 0.6-1.2° C., even more particularly when combined with 2′-MOE modifications.

Another modification of the oligomers of the invention involves chemically linking to the oligomer one or more moieties or conjugates that enhance the activity, cellular distribution or cellular uptake of the oligomer. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl- rac-glycerol or triethylammonium 1,2-di-O-hexadecyl-rac-glycero-3-H-phosphonate, a polyamine or a polyethylene glycol chain, or adamantane acetic acid, a palmityl moiety, myristyl, or an octadecylamine or hexylamino-carbonyl-oxycholesterol moiety.

Cell penetrating peptides have been added to phosphorodiamidate morpholino oligomers to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629.

It is not necessary for all positions in a given compound to be uniformly modified, and in fact more than one of the aforementioned modifications may be incorporated in a single compound or even at a single nucleoside within an oligomer. The present invention also includes AONs that are chimeric compounds. “Chimeric” AONs or “chimeras,” in the context of this invention, are AONs, particularly oligomers, which contain two or more chemically distinct regions, each made up of at least one monomer unit, i.e., a nucleotide in the case of an oligomer compound. These oligomers typically contain at least one region wherein the oligomer is modified so as to confer upon the oligomer or AON increased resistance to nuclease degradation, increased cellular uptake, and an additional region for increased binding affinity for the target nucleic acid.

The activity of AONs and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

The present invention provides AON induced splice-switching of the ACE2 gene transcript, clinically relevant oligomer chemistries and delivery systems to direct ACE2 splice manipulation to therapeutic levels. Substantial decreases in the amount of full length ACE2 mRNA, and hence full length ACE2 protein from ACE2 gene transcription, are achieved by:
a) oligomer refinement in vitro using fibroblast cell lines, through experimental assessment of (i) intronic-enhancer target motifs, (ii) AON length and development of oligomer cocktails, (iii) choice of chemistry, and (iv) the addition of cell-penetrating peptides (CPP) to enhance oligomer delivery; and
b) detailed evaluation of a novel approach to generate ACE2 transcripts with one or more missing exons.

As such, it is demonstrated herein that alternative splicing of ACE2 pre-mRNA can be modulated with specific AONs. In this way functionally significant decreases in the amount of full length (capable of virus internalisation) ACE2 protein can be obtained, and/or increases in the soluble decoy-receptor ACE2 isoforms or other decoy receptors can be achieved, thereby reducing the severe pathology associated with diseases such as such as coronavirus infection, lung diseases, chronic heart disease, chronic kidney disease, hypertension and diabetes.

The AONs used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs of the invention are synthesised in vitro and do not include antisense compositions of biological origin, or genetic vector constructs designed to direct the in vivo synthesis of AONs. The molecules of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

The AONs of the present invention also can be used as a prophylactic or therapeutic, which may be utilised for the purpose of treatment of a disease. Accordingly, in one embodiment the present invention provides AONs that bind to a selected target in the ACE2 pre-mRNA to induce efficient and consistent exon skipping as described herein, in a therapeutically effective amount, admixed with a pharmaceutically acceptable carrier, diluent, or excipient.

The invention therefore provides a pharmaceutical, prophylactic, or therapeutic composition to treat, prevent or ameliorate the effects of a disease associated with ACE2 expression in a subject, the composition comprising:
a) one or more AONs as described herein, and
b) one or more pharmaceutically acceptable carriers and/or diluents.

Preferably the disease associated with ACE2 expression is chosen from the list comprising: coronavirus infection; cardiovascular disorders; respiratory disorders, musculoskeletal, kidney disorders, and endocrine disorders.

In one form of the invention, the ACE2-related disorder is a coronavirus infection selected from the group: Severe acute respiratory syndrome-related coronavirus, SARS, SARS-2, HNL63-CoV (NL63-S) and WIV1-CoV.

In one form of the invention, the ACE2-related disorder is a cardiovascular disorder selected from the group: atherosclerosis, ischaemic heart disease, myocarditis, endocarditis, cardiomyopathy, acute rheumatic fever, chronic rhematic heart disease, cerebrovascular disease/stroke, heart failure, vascular calcification, peripheral vascular disease, and lymphangitis.

In one form of the invention, the ACE2-related disorder is a respiratory (pulmonary) disorder and is selected from the group: acute upper respiratory infections, rhinitis, nasopharyngitis, sinusitis, laryngitis, influenza and pneumonia, acute bronchitis, acute bronchiolitis, asthma, chronic obstructive pulmonary disease (COPD), bronchiectasis, emphysema, chronic lung diseases due to external agents, Acute Respiratory Distress Syndrome (ARDS), pulmonary eosinophilia, and pleuritic, lung trauma and recovery from lung injury, trauma or surgery.

In one form of the invention, the ACE2-related disorder is a kidney disorder and is selected from the group: glomerulonephritis, nephritis, diabetic kidney disease, interstitial nephritis, obstructive and reflux nephropathy, acute renal failure, and chronic kidney disease.

In one form of the invention, the ACE2-related disorder is an endocrine disorder selected from the group: diabetes mellitus, insulin resistance, impaired glucose tolerance and thyroiditis.

The composition may comprise about 1 nM to 1000 nM of each of the desired AON(s) of the invention. Preferably, the composition may comprise about 1 nM to 500 nM, 10 nM to 500 nM, 50 nM to 750 nM, 10 nM to 500 nM, 1 nM to 100 nM, 1 nM to 50 nM, 1 nM to 40 nM, 1 nM to 30 nM, 1 nM to 20 nM, most preferably between 1 nM and 10 nM of each of the AON(s) of the invention.

The composition may comprise about 1 nm, 2 nm, 3 nm, 4 nm, 5 nm, 6 nm, 7 nm, 8 nm, 9 nm, 10 nm, 20 nm, 50 nm, 75 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm or 1000 nm of each of the desired AON(s) of the invention.

The present invention further provides one or more AONs adapted to aid in the prophylactic or therapeutic treatment, prevention or amelioration of symptoms of a disease such as a ACE2 expression related disease or pathology in a form suitable for delivery to a subject.

The phrase “pharmaceutically acceptable” refers to molecular entities and compositions that are physiologically tolerable and do not typically produce an allergic or similarly untoward reaction, such as gastric upset and the like, when administered to a subject. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

In a more specific form of the invention there are provided pharmaceutical compositions comprising therapeutically effective amounts of one or more AONs of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol).The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, for example, Martin, Remington's Pharmaceutical Sciences, 18th Ed. (1990, Mack Publishing Co., Easton, PA 18042) pages 1435-1712 that are herein incorporated by reference. The compositions may be prepared in liquid form, or may be in dried powder, such as a lyophilised form.

It will be appreciated that pharmaceutical compositions provided according to the present invention may be administered by any means known in the art. Preferably, the pharmaceutical compositions for administration are administered by injection, orally, topically or by the pulmonary or nasal route. The appropriate route may be determined by one of skill in the art, as appropriate to the condition of the subject under treatment.

In certain embodiments, the AONs of the disclosure can be delivered by pulmonary or nasal routes (e.g., via nebulised saline incorporating the AONs). High levels of endogenous expression of ACE2 mRNA are found in the lung and is accessible via the airways. Inhaled oligonucleotides are an emerging therapeutic modality for respiratory diseases. The airways are uniquely lined with pulmonary surfactants, which are primarily composed of zwitterionic lipids. These surfactant lipids possess cationic properties at the pH of the respiratory tract. When anionic oligonucleotides are inhaled, they tend to be adsorbed by the surfactants, resulting in reformulated particles that have been hypothesised to be efficiently taken up by bronchial and alveolar epithelial cells into the pulmonary cells. Of note, AONs have been shown to be able to withstand the nebulization process.

In certain embodiments, the AONs are more preferably delivered by intravenous, intra-arterial, intraperitoneal, intramuscular or subcutaneous routes of administration. Vascular or extravascular circulation, the blood or lymph system, and the cerebrospinal fluid are some non-limiting sites where the AON may be introduced.

In certain embodiments, direct CNS delivery may be employed, for instance, intracerebral, ventricular or intrathecal administration may be used as routes of administration.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the AONs of the disclosure can be delivered by transdermal methods (e.g., via incorporation of the AONs into, e.g., emulsions, with such AONs optionally packaged into liposomes). Such transdermal and emulsion/liposome-mediated methods of delivery are described for delivery of AONs in the art, e.g., in U.S. Pat. No. 6,965,025.

The AONs described herein may also be delivered via an implantable device. Design of such a device is an art-recognized process, with, e.g., synthetic implant design described in, e.g., U.S. Pat. No. 6,969,400.

Compositions and formulations for oral administration include powders or granules, microparticulates, nanoparticulates, suspensions or solutions in water or non-aqueous media, capsules, gel capsules, sachets, tablets or minitablets. Thickeners, flavouring agents, diluents, emulsifiers, dispersing aids or binders may be desirable. Oral formulations are those in which oligomers of the disclosure are administered in conjunction with one or more penetration enhancers surfactants and chelators. Surfactants include fatty acids and/or esters or salts thereof, bile acids and/or salts thereof. Bile acids/salts and fatty acids and their uses are further described in U.S. Pat. No. 6,287,860. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Oligomers of the disclosure may be delivered orally, in granular form including sprayed dried particles, or complexed to form micro or nanoparticles. Oligomer complexing agents and their uses are further described in U.S. Pat. No. 6,287,860. Oral formulations for oligomers and their preparation are described in detail in U.S. Pat. No. 6,887,906, Ser. No. 09/315,298 filed May 20, 1999 and/or US20030027780.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833.0ther methods of delivery of AONs to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

It may be desirable to deliver the AON in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods.lt has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the AON of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is a derivative with one or more hydrophilic polymers, such as a polyethylene glycol (PEG) moiety. Liposomes and their uses are further described in U.S. Pat. No. 6,287,860.

The AONs described herein may also be delivered via an implantable device. Design of such a device is an art-recognized process, with, e.g., synthetic implant design described in, e.g., U.S. Pat. No. 6,969,400, the contents of which are incorporated in their entirety by reference herein.

Antisense oligonucleotides can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, AONs may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The AON may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281 ; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813; Barteau et al. (2008), Curr Gene Ther; 8(5):313-23; Mueller et al. (2008). Clin Rev Allergy Immunol; 35(3):164-78; Li et al. (2006) Gene Ther., 13(18):1313-9; Simoes et al. (2005) Expert Opin Drug Deliv; 2(2):237-54.

The AONs of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e. salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be topical (including ophthalmic and mucous membranes, as well as rectal delivery), pulmonary, e.g., by inhalation or insufflation of powders or aerosols (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Oligomers with at least one 2′-MOE modification are believed to be particularly useful for oral administration. Preferably, the AON is delivered via the pulmonary route.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s),In general the formulations are prepared by uniformly and intimately bringing into association the active ingredients with liquid carriers or finely divided solid carriers or both, and then, if necessary, shaping the product.

In one embodiment, the AON is administered in an amount and manner effective to result in a peak blood concentration of at least 200-400 nM AON. Typically, one or more doses of AON are administered, generally at regular intervals, for a period of about one to two weeks. Preferred doses for oral administration are from about 1 mg to 1000 mg oligomer per 70 kg. In some cases, doses of greater than 1000 mg oligomer/subject may be necessary. For i.v. administration, preferred doses are from about 0.5 mg to 1000 mg oligomer per 70 kg. For intra venous or sub cutaneous administration, the AON may be administered at a dosage of about 120 mg/kg daily or weekly.

The AON may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the subject. Persons of ordinary skill can easily determine optimum dosages, dosing methodologies and repetition rates. Optimum dosages may vary depending on the relative potency of individual oligomers, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligomer is administered in maintenance doses, ranging from 0.01 μg to 100 g per kg of body weight, once or more daily, to once every 20 years.

An effective in vivo treatment regimen using the AONs of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered AONs of the invention may be determined from biological samples (tissue, blood, urine etc.) taken from a subject prior to, during and subsequent to administration of the AON. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

Intranuclear oligomer delivery is a major challenge for AONs. Different cell-penetrating peptides (CPP) localize PMOs to varying degrees in different conditions and cell lines, and novel CPPs have been evaluated by the inventors for their ability to deliver PMOs to the target cells. The terms CPP or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration. CPPs are well-known in the art and are disclosed, for example in U.S. Application No. 2010/0016215, which is incorporated by reference in its entirety.

The present invention therefore provides AONs of the present invention win combination with cell-penetrating peptides for manufacturing therapeutic pharmaceutical compositions.

According to a still further aspect of the invention, there is provided one or more AONs as described herein for use in an AON-based therapy. Preferably, the therapy is for a condition related to ACE2 expression. More preferably, the therapy for a condition related to ACE2 expression is therapy for a disease chosen from: coronavirus infection, lung disorders, chronic kidney disease, chronic heart disease hypertension and diabetes.

More specifically, the AON may be selected from the group consisting of any one or more of the AONs listed in table 3 and/or SEQ ID NOs: 1-31, more preferably SEQ ID NO: 5, 6, 9 or 11 and combinations or cocktails thereof. More preferably, the AON is SEQ ID NO: 6 & 9 or SEQ ID NO: 6 and 11. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a ACE2 gene transcript.

The invention extends also to a combination of two or more AONs capable of binding to a selected target to induce exon exclusion in a ACE2 gene transcript. The combination may be a cocktail of two or more AONs, a construct comprising two or more or two or more AONs joined together for use in an AON-based therapy. The combination of AONs is preferably a combination of SEQ ID NO: 6 and 9, or a combination of SEQ ID NO: 6 and 11.

The invention provides a method to treat, prevent or ameliorate the effects of a disease associated with ACE2 expression, comprising the step of:
a) administering to the subject an effective amount of one or more AONs or pharmaceutical composition comprising one or more AONs as described herein.

Furthermore, the invention provides a method to treat, prevent or ameliorate the effects of coronavirus infection, lung disorders, chronic kidney disease, chronic heart disease hypertension and diabetes comprising the step of:
a) administering to the subject an effective amount of one or more AONs or pharmaceutical composition comprising one or more AONs as described herein.

Preferably, the therapy is used to increase soluble levels of ACE2 protein via an exon skipping strategy. The increasing in levels of ACE2 is preferably achieved by modulating the transcripts level through modifying pre-mRNA splicing in the ACE2 gene transcript or part thereof.

The increase in soluble ACE2 will preferably lead to a reduction in the quantity, duration or severity of the symptoms of a ACE2-related condition or pathology, such as coronavirus infection, lung disorders, chronic kidney disease, chronic heart disease, hypertension and diabetes.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

According to another aspect of the invention there is provided the use of one or more AONs as described herein in the manufacture of a medicament for the modulation or control of a disease associated with ACE2 expression.

The invention also provides for the use of purified and isolated AONs as described herein, for the manufacture of a medicament for treatment of a disease associated with ACE2 expression.

There is provided the use of purified and isolated AONs as described herein for the manufacture of a medicament to treat, prevent or ameliorate the effects of a disease associated with ACE2 expression.

Preferably, the ACE2-related pathology or disease is coronavirus infection, lung disorders, chronic kidney disease, chronic heart disease hypertension and diabetes.

The invention extends, according to a still further aspect thereof, to cDNA or cloned copies of the AON sequences of the invention, as well as to vectors containing the AON sequences of the invention. The invention extends further also to cells containing such sequences and/or vectors.

The AON of the present invention may be co-administered with another therapeutic molecule. For example, the AON may be administered with a second therapeutic agent that is a compound, such as a blocker of the renin angiotensin system, such as an ACE inhibitor or Angiotensin Receptor Blocker. Anti-inflammatory agents may also be provided in combination with the AONs of the present invention.

In one form of the invention, AONs are co-administered with other agents that modulate coronavirus infectivity, including passive immunisation and antiviral therapy. If the ACE2-related pathology or disease is coronavirus infection, the AON of the present invention may be co-administered with another antiviral therapeutic molecule chosen from the list comprising: oseltamivir (Tamiflu®), zanamivir (Relenza®), ribavirin, remdesivir, penciclovir, faviparvir, nafamostat, nitazoxanide, camostat mesylate, interferon a (e.g. , interferon α B2), ritonavir, lopinavir, ASC09, azvudine, baloxavir marboxil, darunavir, cobicistat, azithromycin, chloroquine and hydroxychloroquine. Such additional therapeutic agents may be of particular assistance if the coronavirus is SARS-CoV-2.

The invention also provides kits to treat, prevent or ameliorate a disease or condition associated with ACE2 expression in a subject, which kit comprises at least an isolated or purified AON for modifying pre-mRNA splicing in a ACE2 gene transcript or part thereof, packaged in a suitable container, together with instructions for its use.

In a preferred embodiment, the kits will contain at least one AON as described herein, any one or more of SEQ ID NOs: 1-31, more preferably SEQ ID NO: 5, 6, 9 or 11, and/or the sequences set forth in any of Table 3, or a cocktail of AONs, as described herein. The kits may also contain peripheral reagents such as buffers, stabilizers, etc. More preferably, the AON is SEQ ID NO: 6 & 9, or SEQ ID NO: 6 and 11.

There is therefore provided a kit to treat, prevent or ameliorate a disease or condition associated with ACE2 expression in a subject, which kit comprises at least an AON described herein, any one or more of SEQ ID NOs: 1-31, more preferably SEQ ID NO: 5, 6, 9 or 11, and/or the sequences set forth in Table 3 and combinations, or cocktails thereof, packaged in a suitable container, together with instructions for its use. More preferably, he combination of AONs is preferably a combination of SEQ ID NO: 6 and 9, or SEQ ID NO: 6 and 11.

Preferably, the disease or condition is chosen from the list comprising: coronavirus infection, lung disorders, chronic kidney disease, chronic heart disease hypertension and diabetes.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

Those of ordinary skill in the field should appreciate that applications of the above method has wide application for identifying AONs suitable for use in the treatment of many other diseases.

The AONs of the present invention may also be used in conjunction with alternative therapies, such as drug therapies.

The present invention therefore provides a method of treating, preventing or ameliorating the effects of a disease or condition associated with ACE2 expression, wherein the AONs of the present invention and administered sequentially or concurrently with another alternative therapy associated with treating, preventing or ameliorating the effects of a disease or condition associated with ACE2 expression. Preferably, the disease or condition is chosen from the list comprising: coronavirus infection, lung disorders, chronic kidney disease, chronic heart disease hypertension and diabetes..

### General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value; however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Sequence identity numbers (“SEQ ID NO:”) containing nucleotide and amino acid sequence information included in this specification are collected at the end of the description and have been prepared using the program Patentln Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively.

An antisense oligomer nomenclature system was proposed and published to distinguish between the different antisense oligomers (see Mann et al., (2002) J Gen Med 4, 644-654). This nomenclature became especially relevant when testing several slightly different antisense oligomers, all directed at the same target region, as shown below:
H # A/D (x:y)
the first letter designates the species (e.g. H: human, M: murine)
“#” designates target exon number
“A/D” indicates acceptor or donor splice site at the beginning/end of the exon, respectively
(x y) represents the annealing coordinates where “−” or “+” indicate intronic or exonic sequences respectively. As an example, A[−6+18] would indicate the last 6 bases of the intron preceding the target exon and the first 18 bases of the target exon. The closest splice site would be the acceptor so these coordinates would be preceded with an “A”. Describing annealing coordinates at the donor splice site could be D[+2−18] where the last 2 exonic bases and the first 18 intronic bases correspond to the annealing site of the antisense oligomer. Entirely exonic annealing coordinates that would be represented by A[+65+85], that is the site between the 65th and 85th nucleotide, inclusive, from the start of that exon.

The following examples more fully describe the manner of using the above-described invention, as well as to set forth the best modes for carrying out various aspects of the invention. It is understood that these methods do not limit the scope of this invention, but rather are presented for illustrative purposes.

### EXAMPLES

In each of the following examples, the following general materials and methods apply, unless the context requires otherwise.

Chinese Hamster Ovary (CHO) cells express recombinant proteins in high levels making them an ideal system to interrogate the effect of C-truncation of ACE2 on the expression, activity and secretion of truncated ACE2. For experiments, CHO cells were grown in F12 media (supplemented with 10% FBS). CHO cells were then transfected with a plasmid encoding truncated ACE2 constructs using Lipofectamine 2000. After 2 days, the media was collected, concentrated using a molecular weight cut off filter and examined by western blot using anti-hACE2 antibody (R&D systems) and ACE2 catalytic activity.

For experiments, Caco-2 cells were cultured in MCDB 131 medium (10% FCS with 10 mM glutamine, EGF and hydrocortisone). Calu-3 cells were cultured in EMEM (10% FCS). HeCAT cells were cultured in 10% FBS DMEM. Primary normal human dermal fibroblasts were propagated in DMEM supplemented with 10% FBS DMEM and HMEC in MCDB131 supplemented with 10 ng/ml epidermal growth factor, 1 μg/ml hydrocortisone, 10% FBS DMEM and 1× glutamine. African green monkey (Chlorocebus sp) derived VeroE6 cells were cultured in αMEM medium.

For transfection of AONs, cells were seeded into 6 or 24-well plates and then transfected using Lipofectamine 3000 reagent (0.15 ul/well of lipofectamine 3000; 0.4 ul/well P3000/well) with either AONs targeting human ACE2 or dose-equivalent non-target control (annealing to exon 9 of human RAGE without altering RAGE splicing). Cells were then incubated with 50-200 μM AON/cationic lipoplexes at 37° C. for 24-72 hrs after which time, cells were lysed, RNA extracted and cDNA generated using either the Cycle Threshold method or the Trizol method. One step RT-PCR was performed using Superscript III [Life Technologies: Carlsbad, CA, USA] and approximately 50 ng of total RNA as a template. PCR products using forward and reverse primers to the ACE2 tail region were fractionated on 2% agarose gels in Tris-Acetate-EDTA buffer and the images captured on gel documentation system [Vilber Lourmat, Eberhardzell, Germany].

To measure the quantitative effect of AONs on splicing of ACE2, gene expression was then determined using quantitative real-time RT-PCR using a probe to exon boundaries of ACE2 mRNA.

Media was also collected and concentrated using a molecular weight cut off filter. The presence of soluble ACE2 was then determined by ELISA and by the presence of ACE2 catalytic activity.

To determine the adsorption of the S1 from SARS-CoV-2 onto the cell-surface of VeroE6 cells, transfected cells were incubated with GFP-labelled S1 in serum free Optimum media for 30 minutes, after which time they were washed with media and green fluorescence detected using a fluorescence plate reader.

A plaque assay was used to determine infection with SARS-Cov-2. In this assay, Y613L-ACE2(19-613) was added to VeroE6 cells (0.002-2 ug/ml) after which SARS-CoV-2 (50 PFU) was added to each well. 30 minutes later, the wells were overlaid with 2X L15 media and agarose (0.9% w/v), and then incubated at 37° C. for 1-3 days before plaques were counted and TCID50 estimated.

For in vivo experiments, male C57bl6 mice were treated under general anaesthetic (Ketamine) with a single intratracheal dose of AON (3 mg/kg in 30 uL of DI water) and then followed for 7 days after which time they were humanely killed using CO2 narcosis. Bronchoalveolar lavage (BAL) was undertaken post-mortem and fluid measured for ACE2 using a commercial ELISA. Lungs were then removed and ACE2 mRNA expression estimated after Trizol extraction.

### Example 1. Construction and Validation of a C-Truncated Ace2 Mutant

This example demonstrates that a C-truncated ACE2 isoform can be soluble, stable, secreted, catalytically-active and retain antiviral activity when expressed in vitro and in vivo.

A series of C-truncated murine ACE2 (mAce2) mutants were first generated and expressed to establish the effect of C-truncation on ACE2 secretion, stability and enzymatic activity in vitro and in vivo. The endogenous N-terminal signal peptide (1-18) was omitted from all constructs and a 6-His tag and a short linker (-GKT)- was added to the C-terminal of all constructs for purification purposes. The 615 site was chosen because it represents the putative boundary with the collectrin-homology domain (Table 1) and is the product if alternative splicing resulted in skipping of exon 14. The 740 site was chose at it represents the boundary with the transmembrane domain. The 697 site was chosen as it represents the putative inducible cleavage site for ACE2. Murine ACE2 was specifically used, as the expression of non-murine ACE2 leads to development of neutralising antibodies that preclude long-term testing in vivo.

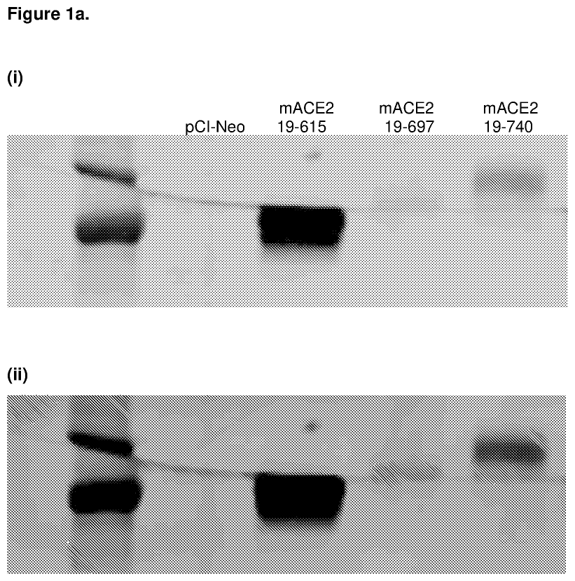

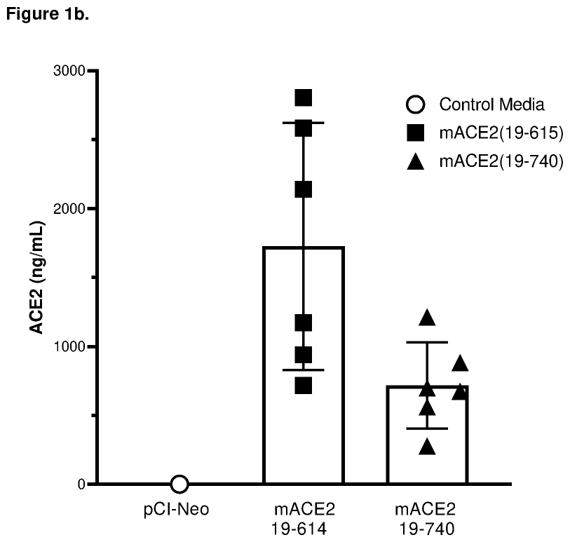

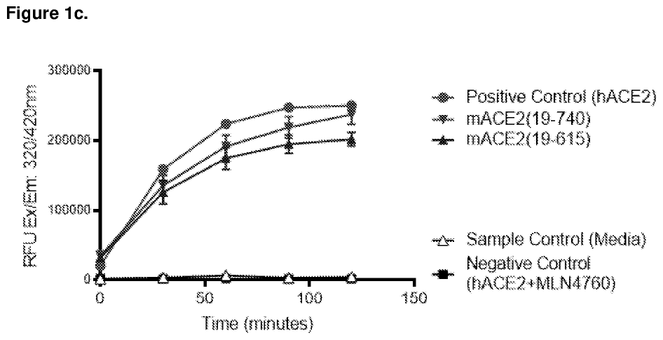

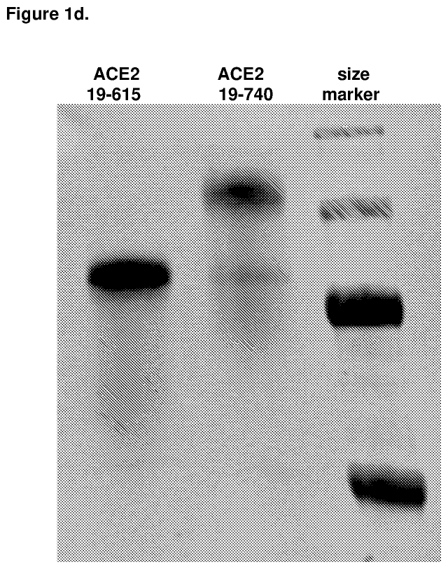

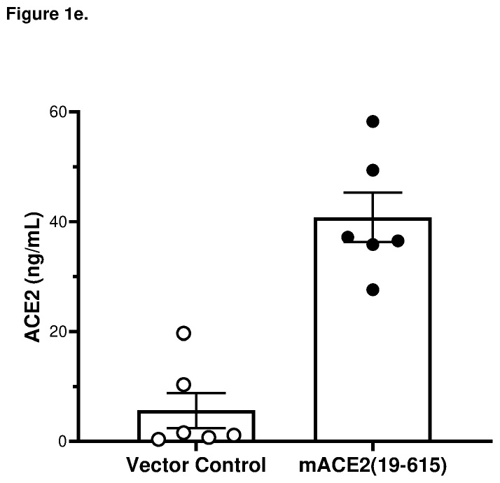

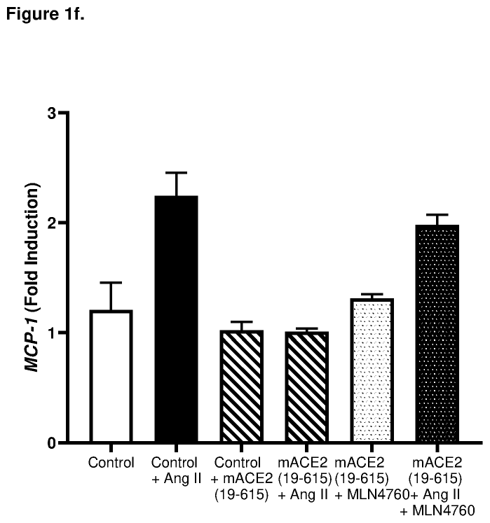

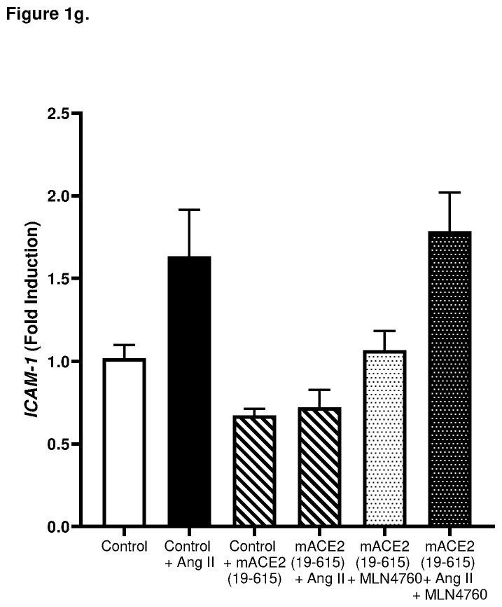

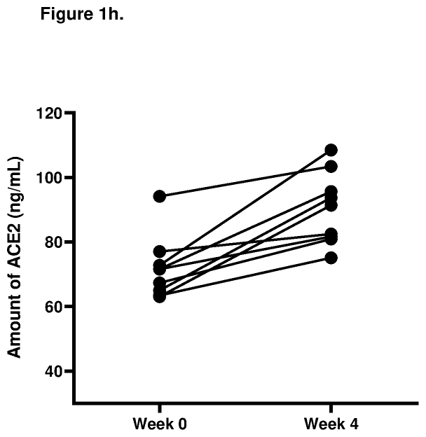

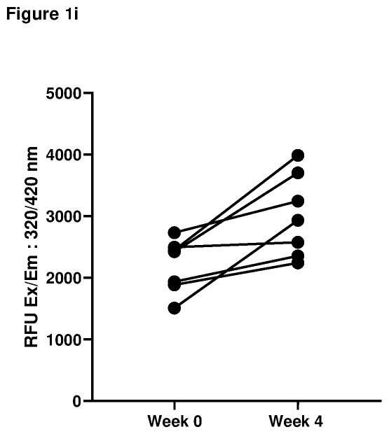

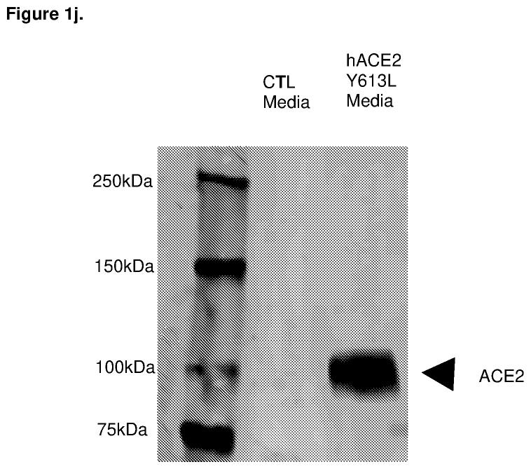

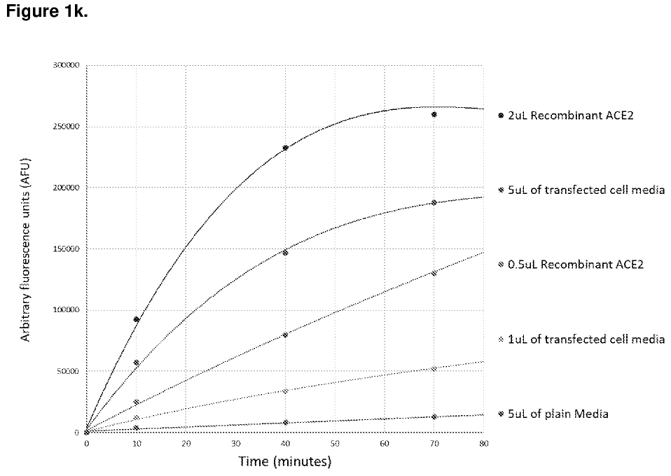

The highest amount of active ACE2 secreted into the media was observed 48 h following transfection of CHO cells with plasmids containing mAce2(19-615), with lesser amounts observed with mAce2(19-740) by western blot (FIG. 1a) and ELISA (FIG. 1b). Moreover, both constructs produced enzymatically activity protein (FIG. 1c) Notably, poor expression was observed with mAce2(19-697), despite being the putative product of inducible ACE2 shedding (FIG. 1a).

The shorter ACE2 construct, mAce2(19-615) was also secreted in higher amounts than mAce2(19-740) when CHO cells were transfected with DNA mini-circles containing C-truncated ACE2, as demonstrated on Western Blotting (FIG. 1d). Transfection of CHO cells with a DNA minicircle encoding mAce2(19-615) also generated ACE2 protein in the media, as demonstrated by ELISA (FIG. 1e).

Media from mAce2(19-615) transfected CHO cells also antagonised Ang II (1 μM) dependent induction of ICAM-1 and MCP-1 gene expression in human aortic endothelial cells, consistent with the presence of functionally relevant angiotensinase activity (FIGS. 1f and 1g). Notably, this protection was blocked by the selective ACE2 inhibitor, MLN-4760, consistent with protection being conferred solely by the ACE2 catalytic activity in the media.

DNA mini-circles encoding mAce2(19-615) were then injected the calf muscle of C57bl6 mice (40 μg/IM) and induced a detectable increase in circulating ACE2 protein and activity 4-weeks post-injection (FIGS. 1h and 1i, respectively), confirming that a C-truncated product mAce2(19-615) can both be expressed, secreted and retain enzymatic function in vivo, consistent with its conformational integrity.

A human analogue of the optimal mAce2(19-615) construct, Y613L-ACE2(19-613) was then developed and transfected into CHO cells, where it also expressed in high levels and generated ACE2 protein that was efficiently secreted into the media (FIG. 1j), with catalytic ACE2 activity comparable to media spiked with commercial recombinant ACE2 (1-740; FIG. 1k).

Plaque accumulation following infection of VeroE6 cells with SARS-CoV-2 was also inhibited following treatment with Y613L-ACE2 protein concentrated from transfected-CHO cell media in a dose-dependent fashion (FIG. 1l) confirming its antiviral activity.

Taken together, this example demonstrates that an ACE2 isoform that is preferentially C-truncated at the putative boundary of the catalytic (ACE-like) and collectrin-like domains would express well, be secreted, enzymatically-active and anti-viral, consistent with conformational integrity.

### Example 2. Modulating the Splicing of Exon 14 of ACE2 Using AONs in Caco-2 Cells

This example details how AONs were designed and utilised to skip exon 14 in human ACE2 pre-mRNA, to generate a novel ACE2 mRNA splice variant (Δ14 splice variant) encoding a soluble ACE2 isoform that is truncated at the putative boundary of the ACE-like and collectrin-like domains, and at the same time, reducing the expression of conventionally-spliced ACE2 mRNA retaining exon-14 and encoding a full length ACE2 isoform that is membrane bound.

Exon 14 of human ACE2 codes for amino acids 613-633 (FIG. 2a). It is a short coding sequence (59 bp) situated between two large introns (>1 kb; table 1b). Due to the different phases of exon 13 and 15, skipping of exon 14 would introduce an in-frame stop codon more than 55 nucleotides from the nearest downstream exon-exon junction. Such premature termination codons (PTC) introduced by aberrant splicing are usually the target of non-sense mediated decay (NMD), to eliminate inefficient transcripts. But while the 55-bp heuristic predicts NMD sensitivity in the majority of cases, a number of exceptions have been reported. We hypothesise that as skipping of exon 14 would generate a splice variant encoding a functional truncated isoform, Y613L-ACE2(19-613) (FIG. 2a), with this truncation serendipitously occurring precisely at the chimeric boundary between the external ACE-like angiotensinase domain and the collectrin-like internal domain of ACE2, this splice variant may escape NMD.

To induce exon skipping, human colonic epithelial cells (Caco-2) were transfected with 2′O-Me PTO antisense oligonucleotides (50-100 μM) targeting the 5′ and/or 3′ splice sites of Exon 14. Caco-2 endogenously express ACE2 in high levels and are known to take up the SARS-CoV-2. In vector treated cells, there are no splice variants of ACE2 mRNA in which exon 14 is skipped (FIG. 2b, panel i). However, 48-hours after transfection with H14A[−17+8] (100 nM; panel ii) or a combination of AONs H14A[−17+8] and H14D[+13−12] (50 nm each, panel iii) resulted in the de novo generation of the Δ14 splice variant, in which exon 14 was skipped and exon 13 and 15 were spliced together, as demonstrated by RT-PCR amplification curves (FIG. 2b).

The increased expression of the Δ14 splice variant following treatment of Caco-2 cells with H14A[−17+8] or combinations including H14A[−17+8], was associated with a concomitant reduction of conventionally-spliced ACE2 mRNA splice variants retaining exon 14 sequences (FIG. 2c), while total ACE2 mRNA expression was not significantly changed (FIG. 2d).

Similar results were observed in Caco-2 cells transfected with ACE2-targetting AONs, including H14A[−17+8] with H14D[+13−12] and H14A[−17+8] with H14D[+9−16], using the Trizol method for RNA purification in order to achieve greater mRNA copy numbers, where ACE2 splice variants containing exon 14 were reduced by over 80% without any reduction in total ACE2 (i.e. ACE2 splice variants containing both exon 13 and 15; FIG. 2e).

In time-course studies, the de novo generation of the Δ14 splice variant was observed after as little as 24 hours after transfection, as demonstrated by RT-PCR amplification curves (FIG. 2f). In particular, a combination of H14A[−17+8] and H14D[+13−12] (panel i) resulted in the de novo generation of the Δ14 splice variant. Again, no novel splice variants were observed following transfection with a control AON (panel ii).

Taken together, this data demonstrates that a novel ACE2 mRNA splice variant in which exon 14 is skipped (the Δ14 splice variant), unexpectedly avoids substantial NMD, and can be induced following treatment with certain AONs.

### Example 3. Modulating the Splicing of Exon 14 of ACE2 Using AONs in VeroE6 Cells

This example details how AONs designed to skip exon 14 of human ACE2 were also able to modulate the splicing of ACE2 in VeroE6 cells and therein inhibit the adsorption of SARS-CoV-2 spike protein onto the cell surface.

Transfection of VeroE6 cells with 2′-O-Me PTO AONs targeting the 5′ and 3′ splice sites of human Exon 14 also modulate ACE2 splicing in monkey-derived VeroE6 cells. In particular, 48-hours after transfection with a combination of H14A[−17+8] and H14D[+13−12] (50 nm each), there was a significant reduction (>95%) of conventionally-spliced ACE2 mRNA splice variants containing Exon 14 (FIG. 3a). At the same time, total ACE2 expression (denoted by ACE2 mRNA splice variants containing both exon 13 and exon 15) was not significantly changed. This implies that exon 14 skipping must also have occurred.

Transfection of VeroE6 cells with 2′-O-Me PTO AONs targeting the 5′ and 3′ splice sites of human Exon 14, specifically H14A[−17+8] and H14D[+13−12], also resulted in an increase in Ace2 activity in concentrated cell media (FIG. 3b), and increased soluble ACE2 protein in the media as measured by ELISA (FIG. 3c), when compared to cells transfected with a non-target AON, consistent with the liberation of a novel soluble ACE2 protein isoform.

Adsorption of the spike glycoprotein (S1) onto VeroE6 cells is dependent on the surface expression of ACE2. Transfection of VeroE6 cells with 2′-O-Me PTO AONs targeting the 5′ and 3′ splice sites of human Exon 14, specifically H14A[−17+8] and H14D[+13−12], also reduced the adsorption of green fluorescent protein (GFP) labelled S1 onto VeroE6 cells (FIG. 3d), demonstrating the anti-viral activity of transfection with AONs targeting Exon 14 of ACE2.

### Example 4. Optomisation of Oligonucleotides Modulating the Splicing of Exon 14 of ACE2

This example details how an AON designed to skip exon 14 of human ACE2 was modified by moving its target sequence by small amounts to improve its efficacy.

Having shown that, on their own, certain AONs, in particular H14A[−17+8], had a modest capacity to modulate the splicing of exon 14 of ACE2 (FIGS. 2b & 2c), additional 2′-O-Me PTO AONs targeting adjacent regions were designed (SEQ ID NOs: 5-8) and tested in human colonic epithelial (Caco-2) cells. Of these constructs, transfection with H14A[−25−1] and H14A[−22+3](SEQ ID NO: 5 and 6 respectively] had the greatest individual effect on inducing exon 14 skipping after 48 hours (FIG. 4a), as denoted by a smaller band (white arrow) on one-step PCR using primers for sequences between exon 13 and exon 15, exceeding the response with H14A[−17+7] (SEQ ID NO: 4) alone.

Transfection of Calu-3 cells with 2′-O-Me phosphorothioate AONs, H14A[−25−1] and H14A[−22+3] (SEQ ID NO: 5 and 6 respectively) also induced exon 14 skipping after 48 hours (FIG. 4b), as denoted by a smaller band (white arrow) on one-step PCR using primers for sequences between exon 13 and exon 15.

Transfection of Calu-3 cells with a vivo-morpholino formulation of H14A[−22+3] (SEQ ID NO: 6) also induced exon 14 skipping after 72 hours (FIG. 6a), as denoted by a smaller band (white arrow) on one-step PCR using primers for sequences between exon 13 and exon 15. Pre-treatment of Calu-3 cells with a vivo-morpholino formulation of H14A[−22+3] (SEQ ID NO: 6) for 72 h also reduced the viral TCID50 in the media after cells were exposed to SARS-CoV-2 (Vic01) in vitro (FIG. 4c), denoting significant inhibition of the infectious activity of the coronavirus arising from exon 14 skipping.

### Example 5. Modulating the Splicing of Exon 17 of ACE2 Using AONs

This example details how AONs were designed and utilised to skip the penultimate exon of ACE2 pre-mRNA, exon 17, thereby removing the coding sequences for residues 706-762 and generating a novel ACE2 splice variant that does not encode the transmembrane domain, and at the same time, reducing the expression of conventionally-spliced ACE2 mRNA retaining exon 17 that encodes an ACE2 isoform is membrane retained and capable of mediating coronavirus adsorption.

15,000 human fibroblasts were transfected with 25-mer 2′-O-Me PTO AONs targeting the 5′ and 3′ splice sites of Exon 17 of ACE2 or adjacent regions with the potential to function as cis-acting sequences to modulate splicing of exon 17 using Lipofectamine 3000. In control treated and untreated (UT) cells, there is no alternative splicing of ACE2 mRNA in which exon 17 is skipped. However, 24-hours after transfection with H17A[−15+10], H17A[−5+20], H17A[−10+15], H17A[+21+45] expression was observed of a novel ACE2 mRNA splice variant in which exon 17 was skipped (Δ17 splice variant), as demonstrated by a new lower band on one-step PCR, using primers located in exon 13 (forward) and exon 18 (reverse) (FIG. 5b).

Transfection of 30,000 human keratinocyte cell line (HaCat) with 20-mer 2′-O-Me PTO AONs targeting one of the 5′ and 3′ splice sites of Exon 17 or adjacent regions with the potential to function as cis-acting sequences to modulate splicing, also generated the novel Δ17 splice-variant, as demonstrated by a new lower band on one-step PCR, using primers located in exon 13 (forward) and exon 18 (reverse) (FIG. 5c)

Transfection of HaCat cells with the three best 2′-O-Me PTO AONs shown to be altering the splicing of Exon 17, H17A[−2+18], H17A[+26+45] and H17A[+21+45] all led to the generation of the novel Δ17 splice-variant, and reduced expression of the conventionally spliced form in which exon 17 was retained, as demonstrated by a new lower band on one-step PCR (FIG. 5d). This demonstrates that shortened derivatives of the preferred 25-mers, H17A[+21+45] (SEQ ID NO: 9) or H17A[−5+20] (SEQ ID NO: 11) are able to achieve similar efficacy in vitro.

Treatment (1 μM) of Caco-2 cells with a vivo-morpholino formulation of H17A[+21+45] also resulted in a marked suppression of conventionally-spliced ACE2 mRNA encoding exon 17 after 48 hours, as detected by real time RT-PCR (FIG. 5e).

Male C57bl6 mice were then treated with M17A[+21+45], an 2′-O-Me PTO AON construct specific to the mouse ACE2 mRNA sequence (SEQ ID NO: 32), at a dose of 3.0 mg/kg delivered intratracheally in DI water. Seven days later there was a significant reduction in the lungs of conventionally-spliced ACE2 mRNA encoding exon 17, without a significant change in total ACE2, denoting the induction of alternative splicing of ACE2 (FIG. 5f). In addition, there was an increase in soluble ACE2 protein in the bronchoalveolar fluid, as measured by ELISA, when compared to mice treated with a non-target AON control (FIG. 5g), consistent with the modulation of ACE2 pre-mRNA splicing to generate a soluble protein isoform.

Pre-treatment of Calu-3 cells for 72 h with a vivo-morpholino formulation of H17A[+21+45] (2 uM) alone did not significantly reduce the viral TCID50 in the media after cells were then exposed to SARS-CoV-2 (Vic01) in vitro (FIG. 5h). This in contrast to H14A[−22+3] detailed in FIG. 4b. This exemplifies that although soluble ACE2 was produced and full length ACE2 mRNA was diminished by treatment with H17A[+21+45], antiviral activity also requires conformational integrity.

### Example 6. Modulating the Splicing of ACE2 Using a Combination of AONs Targeting Exon 14 and 17 of Human ACE2

This example details how certain AONs can be combined to enhance the generation of novel ACE2 mRNA splice variants that encode and generate a preferred soluble ACE2 isoform, and at the same time, reduce the expression of conventionally-spliced ACE2 mRNA that encodes full-length ACE2, that is both membrane-retained and capable of mediating virus adsorption and uptake.

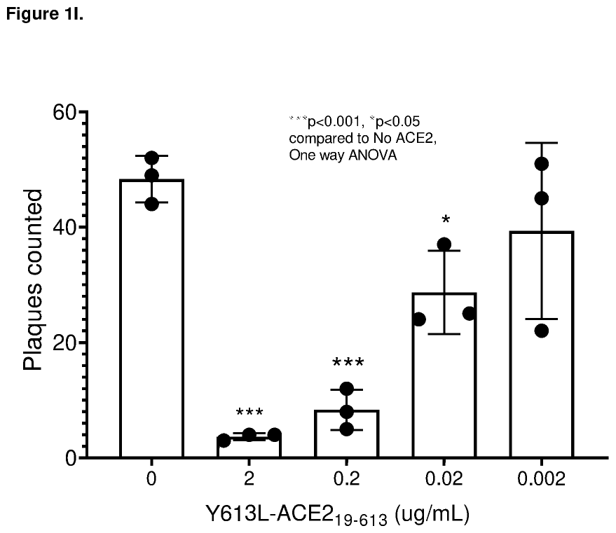

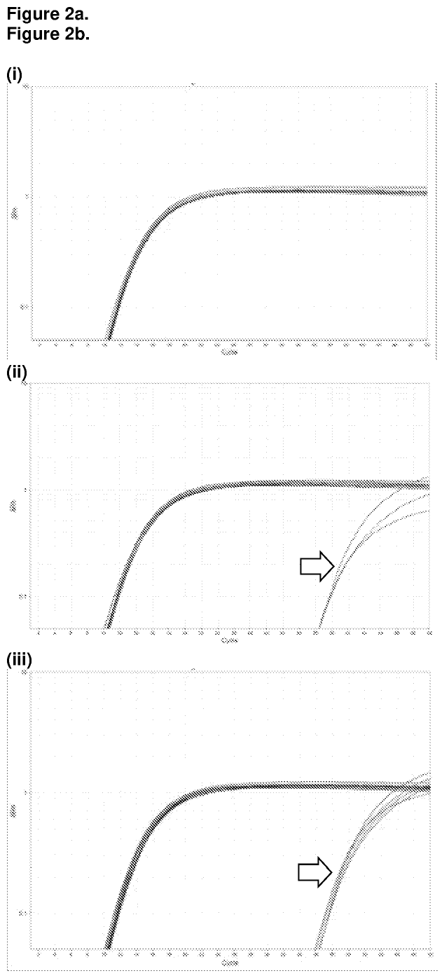

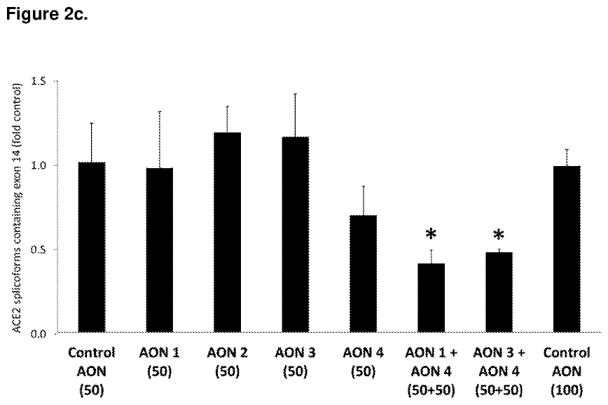

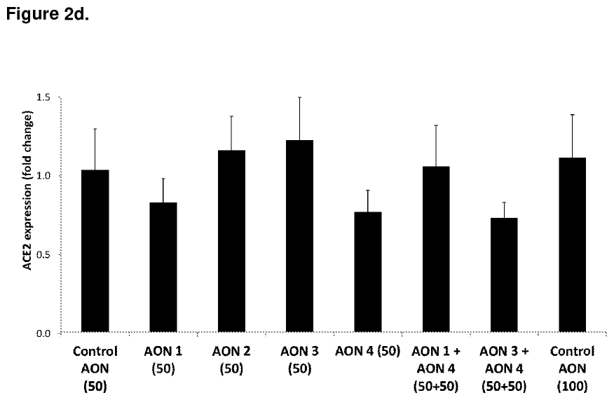

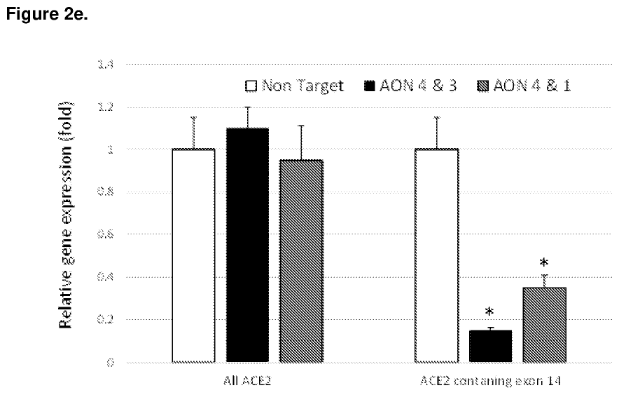

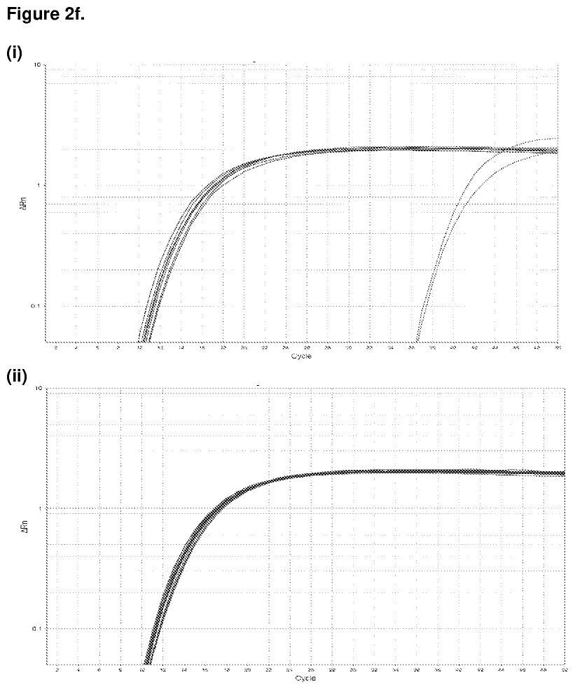

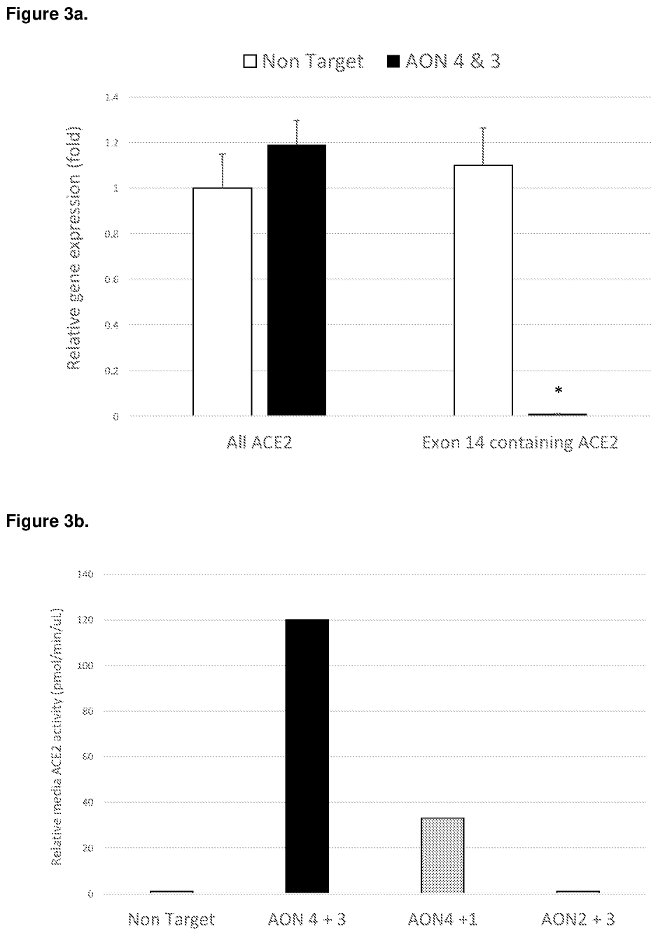

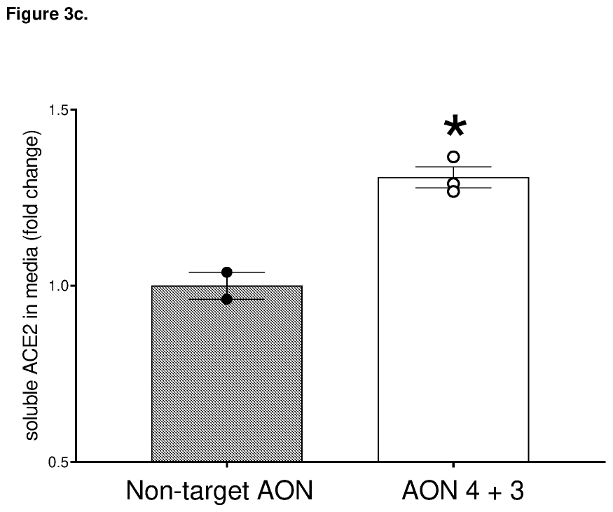

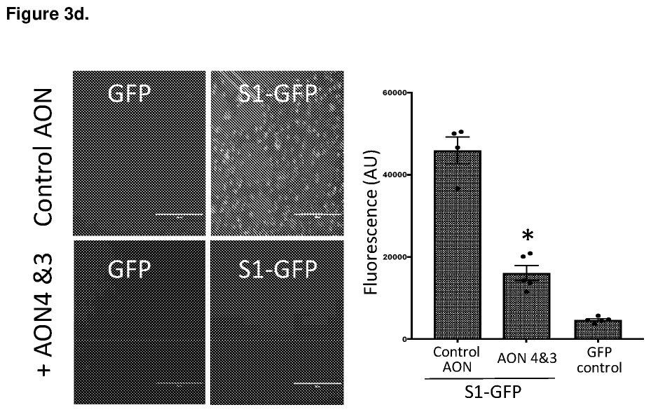

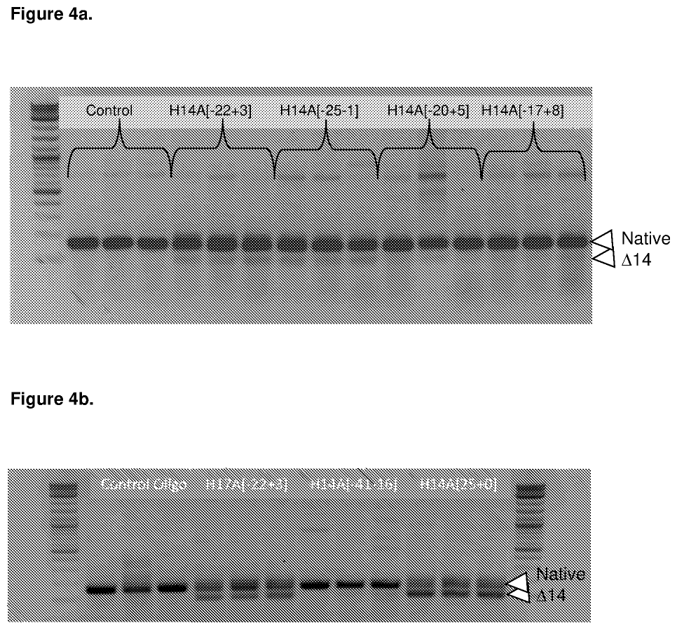

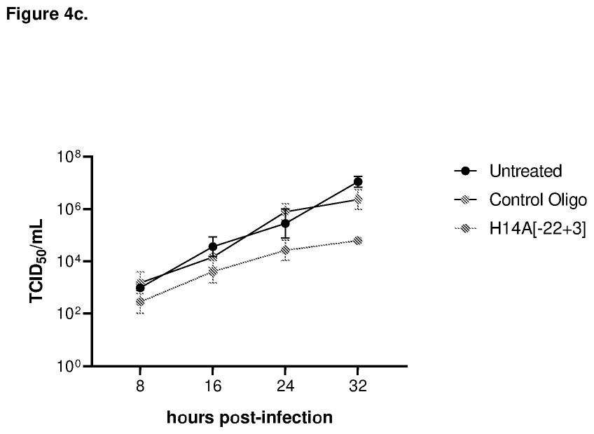

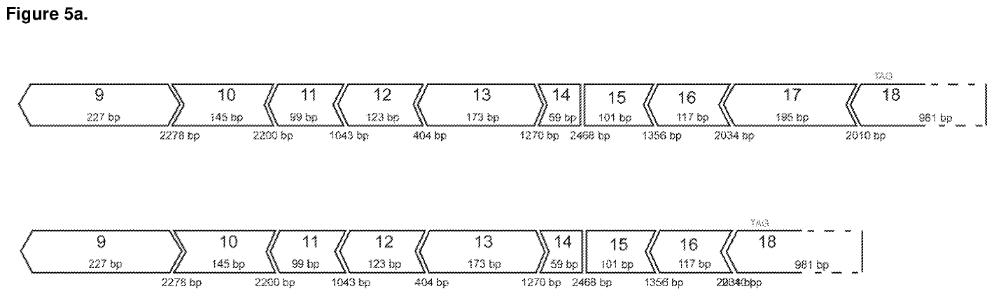

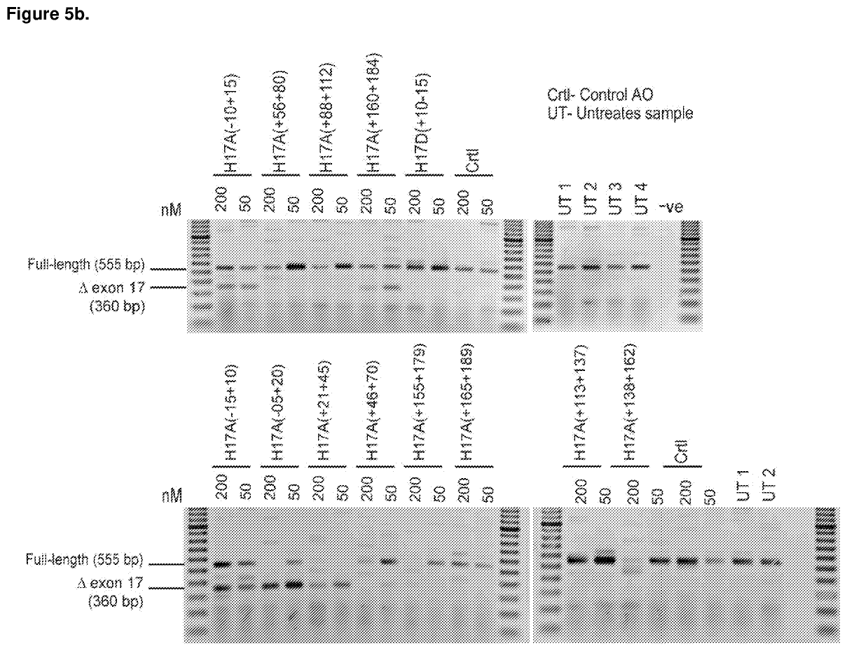

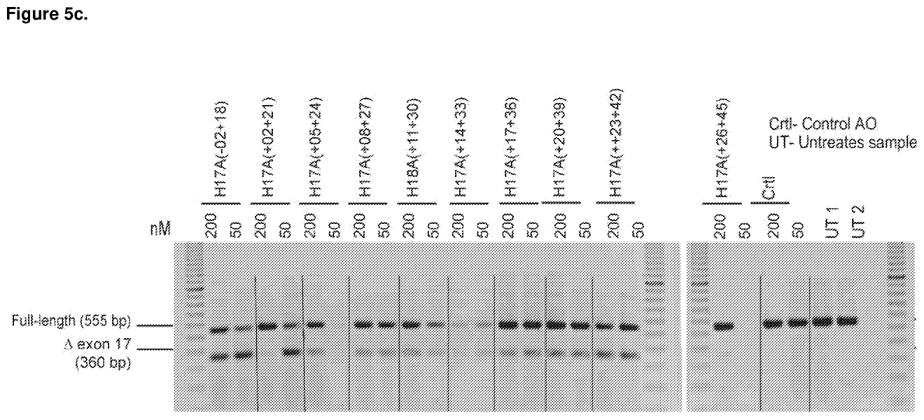

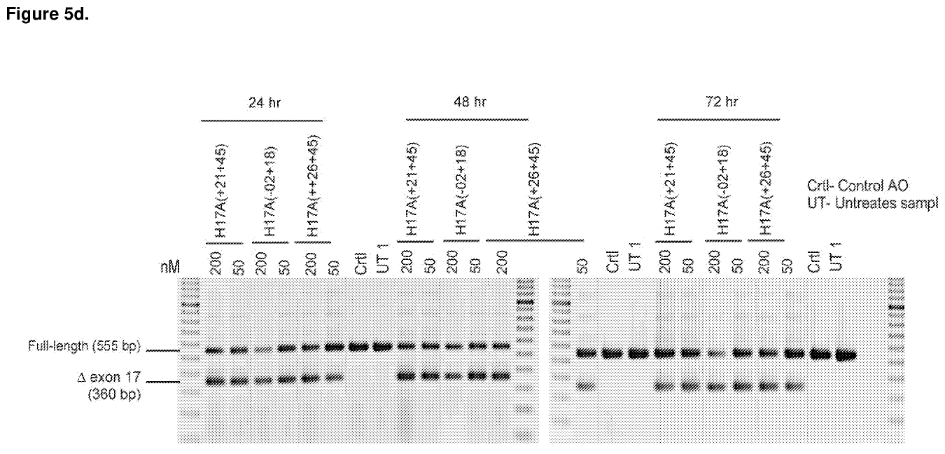

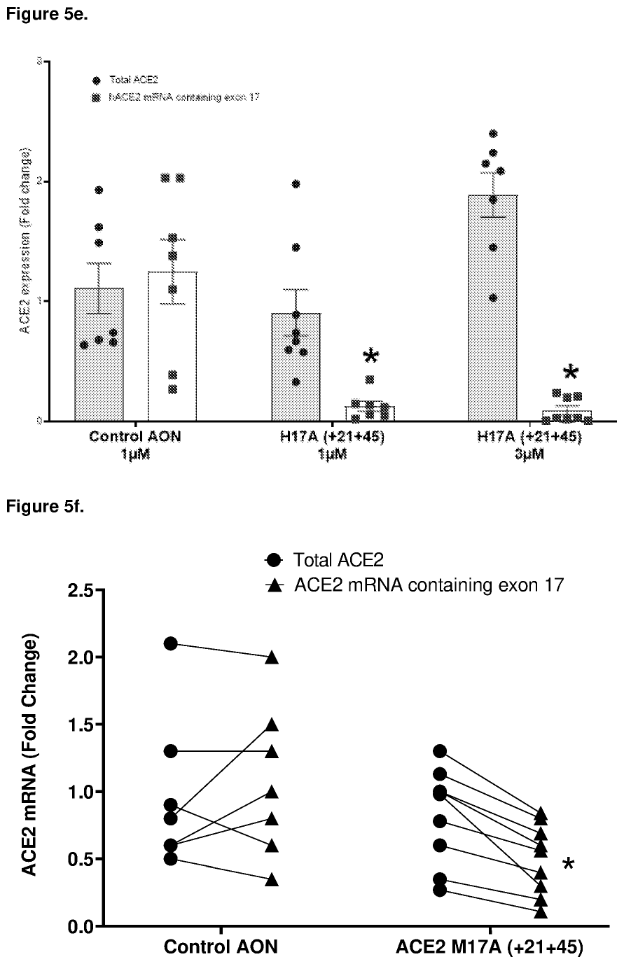

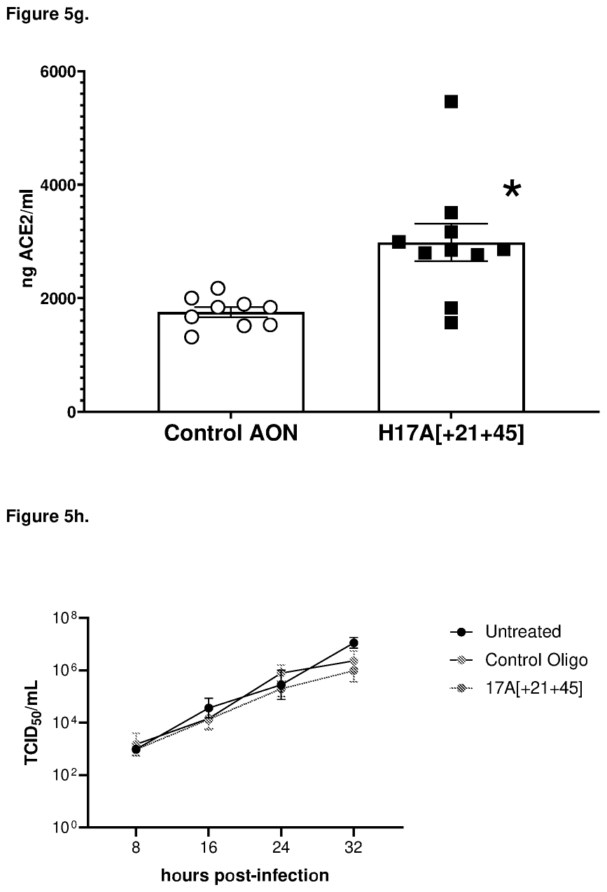

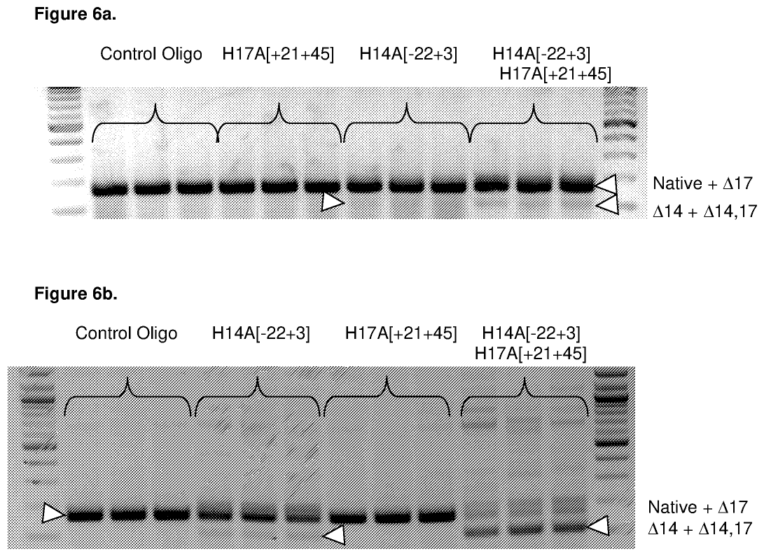

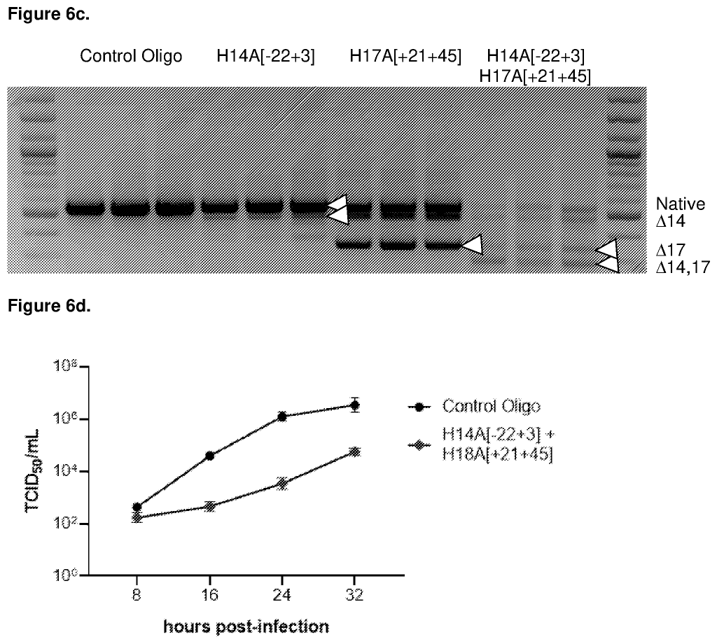

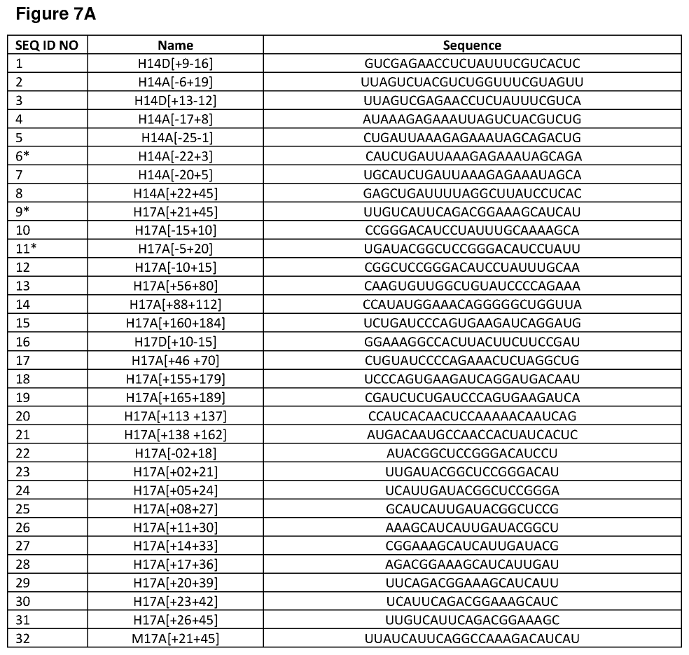

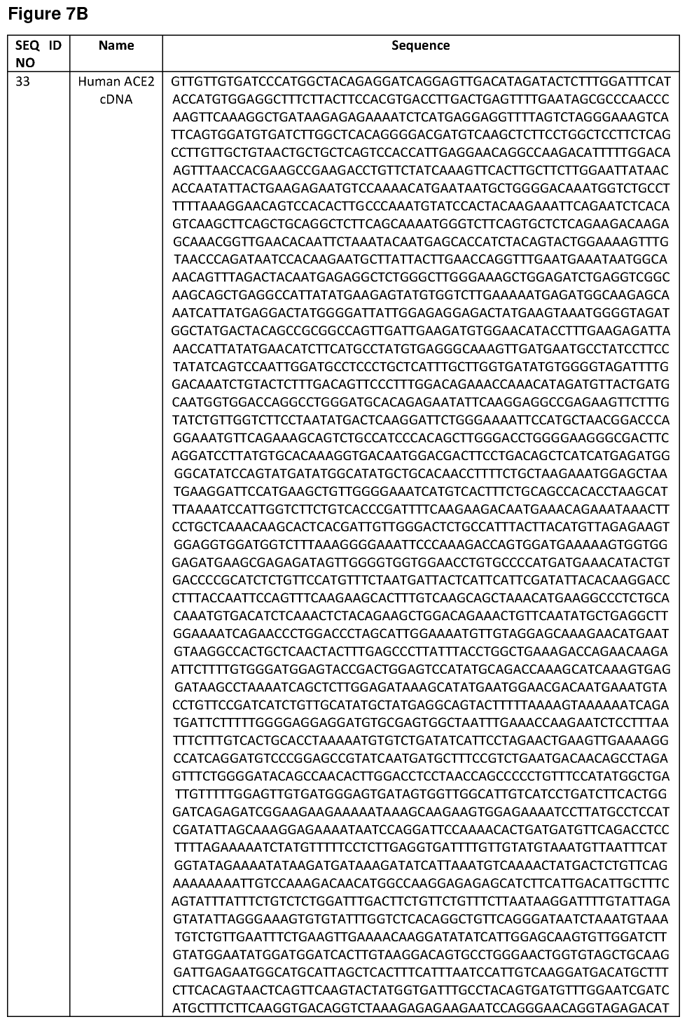

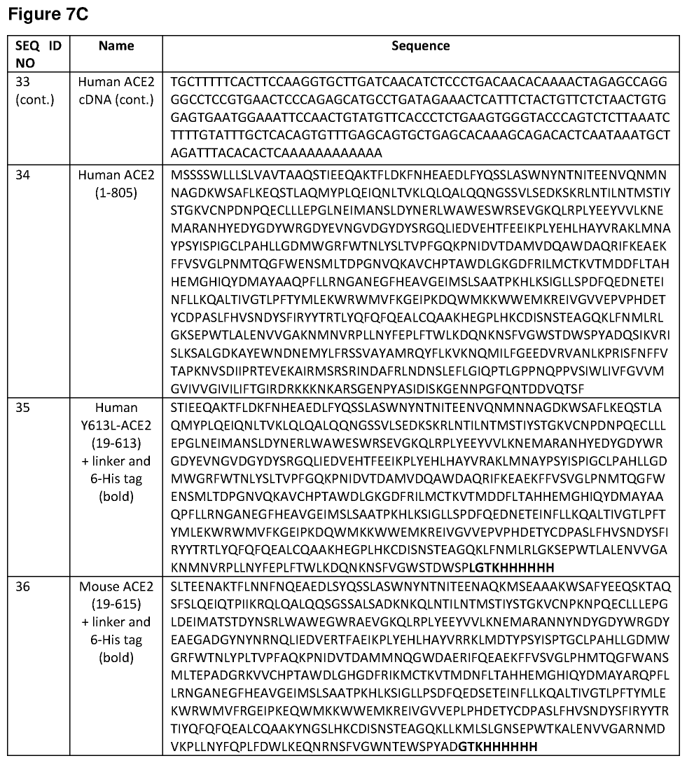

Skipping Exon 17 (e.g. with H17A[+21+45] or H17A[−5+20]) would be expected to generate an ACE2 protein isoform containing residues 19-705, skipping the transmembrane domain, followed by the short (43aa) cytosolic tail of ACE2. It is possible that the protein expression from this novel Δ17 splice variant may be less than Δ14 splice variant which generates Y613L-ACE2(19-613), which we show is more active and expresses better than longer ACE2 ectodomain constructs, including ACE2(19-697) (FIG. 1a). It is also possible that the soluble protein generated by Δ17 splice variant has a different conformation from native ACE2 due to retention of the cytosolic tail that has the potential to interferes with its activity, retention or stability. This is unlike the preferred isoform, Y613L-ACE2(19-613) that is catalytically active and demonstrably antiviral (FIG. 11). We hypothesised that if both exon 14 and 17 could be skipped in ACE2 mRNA (e.g. with H14A[−22+3] used in combination with H17A[+21+45]), it could increase generation of the more-desirable high-expressing Y613L-ACE2(19-613) isoform. In addition, using one AON to alter the splicing of pre-mRNA at one target site also has the potential to modulate the ability of a different AON to induce alternative splicing elsewhere, by changing the conformation of the pre-mRNA (e.g. to increase access of the SSOs to a target sequence, altering the stability or decay of novel splice variants. However, such interactions are unpredictable and unique to each pre-mRNA and each target.

In this example, we show that treatment of Calu-3 cells with a combination of two vivo-morpholino AONs, H17A[+21+45] and H14A[−22+3] (1 μM each), significantly increased the expression of novel ACE2 mRNA splice variants in which exon 14 was skipped, when compared to H14A[−22+3] alone, that had only a modest effect after 72-hours in this cell line (white arrow; FIG. 6a). H17A[+21+45] alone had no effect on exon 14 splicing on its own (as it targets exon 17). The expression of ACE2 mRNA splice variants retaining exon 14 were also reduced (red arrow), 72-hours following treatment with a combination of H17A[+21+45] and H14A[−22+3], as detected by one-step-PCR, using primers located in exon 13 (forward) and exon 15 (reverse).

A similar synergistic response to a combination of two vivo-morpholino AONs, H17A[+21+45] and H14A[−22+3] was also observed in Caco-2 cells, with significantly increased expression of novel ACE2 mRNA splice variants in which exon 14 had been skipped (white arrow; FIG. 6b). Notably, the Δ17 splice variant observed when using H17A[+21+45] alone was also reduced when used in combination with H14A[−22+3] (FIG. 6c), confirming the major splice variant produced by the combination had skipped both exons 17 and 14 (green arrow), as measured by one-step PCR, using primers located in exon 13 (forward) and exon 18 (reverse). This novel splice variant (Δ14,17) would code for the preferred protein product, Y613L-ACE2(19-613). In addition, the reduction in full-length splice variant (red arrow) is clearly greatest with the combination treatment.

A strong antiviral response was observed when pre-treating Calu-3 cells with a combination of vivo-morpholino formulations of H17A[+21+45] and H14A[−22+3] (1 μM each) for 72 hours, as demonstrated by a reduction in viral TCID50 in the media after cells were exposed to SARS-CoV-2 (Vic01) (FIG. 6d), exceeding the response with either AON alone.

1. An isolated or purified antisense oligomer for modifying pre-mRNA splicing in the Angiotensin Converting Enzyme 2 (ACE2) to modulate splicing of the ACE2 gene transcript or part thereof which has a modified backbone structure and sequences with at least 75% sequence identity to such antisense oligomers and which have a modified backbone structure.

2. The antisense oligomer of claim 1 that is chosen from the list comprising:
a) SEQ ID NO: 1-31; and/or
b) SEQ ID NO: 5, 6, 9 or 11; and/or
c) Table 3.

3. The antisense oligomer of claim 1 wherein the antisense oligomer contains one or more nucleotide positions subject to an alternative chemistry or modification chosen from the list comprising: (i) modified sugar moieties; (ii) resistance to RNase H; and/or (iii) oligomeric mimetic chemistry.

4. The antisense oligomer of claim 1 wherein the antisense oligomer is further modified by: (i) chemical conjugation to a moiety; and/or (ii) tagging with a cell penetrating peptide.

5. The antisense oligomer of claim 1 wherein, if a uracil is present in the antisense oligomer, the uracil (U) of the antisense oligomer is replaced by a thymine (T).

6. The antisense oligomer of claim 1 wherein the modification of pre-mRNA splicing results in skipping of one or more exonic sequences of the ACE2 pre-mRNA.

7. A pharmaceutical composition to treat or ameliorate the effects of a disease related to ACE2 expression in a subject, the composition comprising:
a) one or more antisense oligomers according to claim 1, and
b) one or more pharmaceutically acceptable carriers and/or diluents.

8. A method for manipulating splicing in an ACE2 gene transcript, the method including the step of:
a) providing one or more of the antisense oligomers according to claim 1 and allowing the oligomer(s) to bind to a target nucleic acid site.

9. A method to modulate the expression, concentration or activity of ACE2 isoforms comprising the step of:
(a) administering to the subject an effective amount of one or more antisense oligomers or pharmaceutical composition comprising one or more antisense oligomers according to claim 1.

10. A method to treat, prevent or ameliorate the effects of a disease associated with ACE2 expression, comprising the step of:
a) administering to the subject an effective amount of one or more antisense oligomers or pharmaceutical composition comprising one or more antisense oligomers according to claim 1.

11. (canceled)

12. A kit to treat, prevent or ameliorate the effects of a disease associated with ACE2 expression in a subject, which kit comprises at least an antisense oligomer according to claim 1 and combinations or cocktails thereof, packaged in a suitable container, together with instructions for its use.

13. The composition of claim 7, wherein the ACE2 expression related disease is chosen from the list comprising: infections caused by Severe Acute Respiratory Syndrome-related coronaviruses, lung disorders, chronic kidney disease, chronic heart disease hypertension and diabetes.

14. The composition of claim 7, wherein the to the antisense oligomer is administered in combination with second therapeutic agent chosen from the list comprising: soluble ACE2 isoforms, a compound able to modulate the RAAS, antiviral therapy or passive immunotherapy targeting coronaviruses. (Currently amended) The composition of claim 7, wherein the to the antisense oligomer is a combination of antisense oligomers.

16. The composition of claim 15 wherein the combination of antisense oligomers is chosen from the combination SEQ ID NO: 6 and 9 or SEQ ID NO: 6 and 11.
